(19) United States
(12) Patent Application Publication
(10) Pub. No.: US 2021/0290576 A1
(43) Pub. Date: Sep. 23, 2021

(54) STRUCTURALLY MODIFIED FATTY ACIDS FOR IMPROVING GLYCEMIC CONTROL AND TREATING INFLAMMATORY BOWEL DISEASE

(71) Applicants: NORTHSEA THERAPEUTICS B.V., Amsterdam (NL); BASF AS, Oslo (NO)

(72) Inventors: David Alan FRASER, Blommenholm (NO); Tore SKJAERET, Oslo (NO); Detlef SCHUPPAN, Mainz (DE)

(21) Appl. No.: 17/057,204

(22) PCT Filed: May 22, 2019
(86) PCT No.: PCT/IB2019/000655

§ 371 Date: Nov. 20, 2020

(30) Foreign Application Priority Data
May 23, 2018 (NO) 20180714

(51) Int. Cl. A61K 31/202; A61K 45/06; A61K 9/00; A61P 3/10; A61P 1/04

(52) U.S. Cl. CPC A61K 31/202; A61K 45/06; A61P 1/04; A61P 3/10; A61K 9/0053

(57) ABSTRACT

The present disclosure provides a compound for use as an activator of enteroendocrine GLP-1 production, improving glycemic control, and treating inflammatory bowel disease, wherein the compound is a structurally modified unsaturated fatty acid with an α-substituent, either alone or in combination with one or more additional therapeutic agents.

This application claims the benefit of priority of Norwegian Patent Application No. 20180714, filed on May 23, 2018. The foregoing application is incorporated herein by reference in its entirety.

### FIELD OF THE INVENTION

The present disclosure provides compounds for use as a stimulator of intestinal enteroendocrine glucagon-like peptide 1 (GLP-1) production, wherein the compound is a structurally modified unsaturated fatty acid with an α-substituent, for use either alone or in combination with one or more additional therapeutic agents. The present disclosure provides compounds for improving glycemic control, including reducing basal and/or postprandial hyperglycemia and increasing postprandial plasma insulin levels. The present disclosure also provides compounds for use in treating inflammatory bowel diseases (IBD), such as Crohn's disease, ulcerative colitis, and indeterminate colitis.

### BACKGROUND TO THE INVENTION

The G-protein coupled receptor GPR40 (also known as free-fatty acid receptor [FFAR]-1) is highly expressed on pancreatic β-cells and responds to ligand binding by improving glucose stimulated insulin secretion (GSIS). GPR40, along with a related receptor GPR120/FFAR4, is also expressed on enteroendocrine cells (specialised cells of the gastrointestinal tract and pancreas with endocrine function) in the intestine and responds to ligand binding by increasing the secretion of incretins, such as glucagon-like peptide 1 (GLP-1). GLP-1 in turn stimulates GSIS and decreases hepatic glucose output. The glucose dependency of insulin secretion makes both GLP-1, and the receptors GPR40 and GPR120, attractive targets for developing therapies with a good safety profile (avoidance of hypoglycaemia) for use in the treatment of type 2 diabetes (T2DM).

The discovery of intestinal enteroendocrine GLP-1 as a mediator of postprandial insulin secretion was preceded by the observation that intravenous glucose delivery did not stimulate the same insulin response as an oral glucose load. Improved glucose tolerance immediately after bariatric surgery (preceding weight-loss) also suggested cells in the distal intestine were actively involved in regulating postprandial glucose tolerance.

The identification of GLP-1 as a pivotal gut-derived incretin regulating glucose tolerance led to the rapid development of parenteral, and more recently, oral GLP-1 therapies for T2DM. However, as GLP-1 is broken down within minutes of release from the gut, oral compounds that inhibit endogenous GLP-1 breakdown, such as dipeptidyl peptidase-4 (DPP-4) inhibitors, and stable, still largely parenterally administered, GLP-1 analogues (short- and long-acting) that resist DPP-4 degradation, have become an effective therapeutic strategy for patients with T2DM. More recently, GPR40 agonists have also been under clinical development, designed to directly stimulate pancreatic β-cell GSIS.

An alternative strategy for increasing endogenous GLP-1 concentrations is targeting intestinal enteroendocrine cells in the small- and large intestine via GPR40 and/or GPR120 with the natural ligands, i.e., free-fatty acids. However, as shown by Morishita et al., J. Control. Release., 2008, 132(2): 99-104, despite the identification of long-chain omega-3 (n-3) fatty acids as ligands for both GPR40 and GPR120 regulating GLP-1 production from enteroendocrine cells in vitro, oral feeding with long-chain n-3 fatty acids is minimally effective in inducing clinically relevant GLP-1 concentrations and/or improving glycemic control in humans. Without being bound by theory, there are likely several reasons for this.

Firstly, as previously mentioned, GLP-1 is rapidly deactivated by DPP-4 in multiple tissues, resulting in a half-life of less than 2 minutes in humans and less in rodents. This stimulated the development of DDP-4 inhibitor drugs to increase GLP-1 half-life.

Secondly, oral fatty acids are primarily absorbed in the upper small intestine, and are thus unable to target the high concentrations of FFARs in the distal small intestine and large intestine. It has further been reported by Morishita et al., J. Control. Release., 2008, 132(2):99-104 that stimulation of intestinal GLP-1 production via eicosapentaenoic acid is site-specific and dependent upon colonic administration, with no effect observed with delivery to either the stomach or jejunum.

Thirdly, studies reported by Christensen et al., Physiol Rep., 2015, 3(9) have shown that the FFAR, GPR40, is primarily activated on the vascular side of the gut lining, not the luminal side. Thus, long chain fatty acids should be absorbed to optimally activate GPR40. However, orally delivered fatty acids are minimally present in their free acid form on the vascular side of the intestine post-absorption, but are instead incorporated into chylomicrons as triglycerides with minimal capacity to bind and activate FFARs.

Finally, it has also been shown by Tunaru et al., Nat Commun., 2018, 9(1):177, that hydroxylated metabolites of GPR40-binding fatty acids are far more potent than their parent compounds as autocrine GPR40 ligands. Thus, we hypothesized that structural modifications that maximise availability of the free fatty-acid form, minimising incorporation into complex lipids and pre-secretory lipoproteins in enterocytes, and preventing their metabolism, could increase the substrate availability for enzymatic modifications and the generation of more potent FFAR ligands.

In addition to its effects on postprandial insulin secretion, studies suggest that GLP-1 also exerts anti-inflammatory effects. Thus, treatments directed to inducing GLP-1 in the intestine may offer some therapeutic benefit to inflammatory bowel diseases (IBD).

Inflammatory bowel diseases (IBDs) are chronic intestinal inflammatory conditions, characterized by uncontrolled inflammation resulting from inappropriate and persistent activation of the mucosal immune system. Uniken Venema et al., J. Pathol. 2017, 241(2):146-158; Huang et al., Am. J. Transl. Res., 2016, 8(6):2490-2497. The hallmark of active IBD is recruitment of inflammatory cells, their infiltration and activation within the intestinal mucosa and lamina propria, and enhanced production of pro-inflammatory mediators. Fakhoury et al., J. Inflamm. Res., 2014, 7:113-120; Xavier et al., Nature, 2007, 448(7152):427-434. IBD can be grossly categorized as ulcerative colitis, with a prominent Th2 T cell response, and Crohn's disease with a prominent Th1 T cell response. While ulcerative colitis is limited to the gut, Crohn's disease can affect both the colon and the small intestine. A third category is indeterminate colitis, which has features of both ulcerative colitis and Crohn's disease, and which affects 10-15% of IBD patients.

Currently, there is no cure for IBDs, and treatment modalities are focused on reduction of the inflammatory process to alleviate the symptoms and to prevent future complications, to improve the patient's quality of life. Pharmaceutical treatment of IBD includes five major categories: anti-inflammatory drugs including biologicals, immunosuppressants, immune modulators, antibiotics, and drugs for symptomatic relief. However, these treatments are often associated with significant side effects and are of limited success in some patients, highlighting the need for novel, therapeutic agents with minimal or no side effects. Ananthakrishnan et al., Inflamm. Bowel Dis., 2017, 23(6):882-893.

Previous efforts for developing such therapeutic agents for IBD having minimal side effects include using oral administration of naturally occurring omega-3 fatty acids. However, these efforts to treat IBD have been unsuccessful or, at best, inconclusive. Lev-Tzion et al., Cochrane Database Syst. Rev., 2014, 28(2):CD006320; Cabre et al., Br. J. Nutri., 2012, Suppl 2:S240-252. This failure may be, at least in part, because, as described above, orally administered omega-3 fatty acids are primarily absorbed in the upper small intestine, and thus may not be able to target fatty-acid receptor rich segments of the lower intestine and colon. Although direct colonic delivery of EPA and DHA are able to induce GLP-1 secretion in rodents, this approach would be inconvenient for patients versus oral dosing. Importantly, the dose of omega-3 fatty acids needed for the desired effects would be excessive, because they are largely incorporated into cellular membranes or are metabolized instead of activating fatty acid receptors.

Based on the above, there is a need for new and alternative ways to activate enteroendocrine GLP-1 production and/or improve glycemic control. There is also a need for orally administered therapeutics with minimal side effects that treat IBD. We hypothesised that specific structural modifications to fatty acids could improve their ability to bind and stimulate intestinal GPR40/120 and/or increase GLP-1 secretion. We hypothesized that these modified fatty acids could improve glycemic control, such as through decreasing basal and/or postprandial glucose levels and/or increasing postprandial insulin levels, and treat IBD.

### BRIEF SUMMARY OF THE INVENTION

The present disclosure provides compounds for use as stimulators of enteroendocrine GLP-1 production, wherein the compounds are unsaturated fatty acids with substituents in the α-position, for use either alone or in combination with one or more additional therapeutic agents. Without being bound by theory, the modified fatty acids may be ligands for GPR40/120 with an improved ability to reach and activate the receptors located in the ileum and large intestine and/or to inhibit DPP-4 activity.

More particularly, the invention provides compounds for use as a potentiator of enteroendocrine GLP-1 production, improving GSIS, promoting satiety, slowing gastric emptying, inhibiting glucose-dependent glucagon secretion, and reducing hepatic glucose production. The disclosure also provides compounds for use in improving glycemic control, including reducing basal and/or postprandial hyperglycemia, and/or increasing postprandial plasma insulin concentrations.

The disclosure further provides compounds for use in treating IBD, such as Crohn's disease, ulcerative colitis, and indeterminate colitis. The disclosure provides compounds for reducing intestinal inflammation in IBD, inducing remission of IBD, maintaining remission of IBD, reducing weight loss in subjects experiencing IBD symptoms, reducing decrease in colon length, reducing intestinal inflammation in subjects with IBD, and/or reducing intestinal injury in subjects with IBD.

In one aspect the invention provides a method for increasing levels of GLP-1 in a subject in need thereof, comprising administering to the subject a pharmaceutically effective amount of a compound of Formula (I). In some embodiments, the invention provides a method for reducing basal and/or postprandial hyperglycemia and/or increasing postprandial plasma insulin concentrations in a subject in need thereof, comprising administering to the subject a pharmaceutically effective amount of a compound of Formula (I). In some embodiments, the invention provides a method for treating IBD in a subject in need thereof, comprising administering to the subject a pharmaceutically effective amount of a compound of Formula (I).

In some embodiments, the compound is administered to the subject optionally in combination with one or more additional active agents.

The compounds of Formula (I) are:

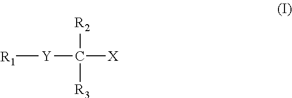

wherein R1 is selected from a 010-C22 alkenyl having 3-6 double bonds;
R2 and R3 are the same or different and are selected from a group of substituents consisting of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group, provided that R2 and R3 can be connected in order to form a cycloalkane like cyclopropane, cyclobutane, cyclopentane or cyclohexane, and provided that both R2 and R3 are not hydrogen;
X is a carboxylic acid or a derivative thereof, wherein the derivative is a carboxylate, such as a carboxylic ester; a glyceride; an anhydride; a carboxamide; a phospholipid; or a hydroxymethyl; or a prodrug thereof;
Y is oxygen, sulphur, sulfoxide, sulfone or CH₂;
or a pharmaceutically acceptable salt, solvate, or solvate of such a salt;
and optionally one or more additional active agents.

In an equal aspect, the invention provides a compound of Formula (I) for use in increasing GLP-1 production in a subject, wherein said compound is administered to the subject optionally in combination with one or more additional active agents.

In some embodiments, the invention provides a compound of Formula (I) for use in improving glycemic control, including reducing basal or postprandial hyperglycemia and/or increasing postprandial plasma insulin concentrations in a subject, wherein said compound is administered to the subject optionally in combination with one or more additional active agents.

In some embodiments, the invention provides a compound of Formula (I) for use in treating IBD in a subject, reducing intestinal inflammation in IBD, inducing remission of IBD, maintaining remission of IBD, reducing weight loss in subjects experiencing IBD symptoms, reducing decrease in colon length, reducing intestinal inflammation in subjects with IBD, and/or reducing intestinal injury in subjects with IBD, wherein said compound is administered to the subject optionally in combination with one or more additional active agents.

More particularly, the compound for use is provided by Formula (II):

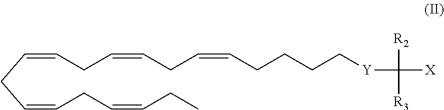

wherein R2, R3, Y and X are defined as for Formula I;

and for administration optionally with one or more additional active agent.

The invention further provides a combination product comprising
i) a first component being a compound of Formula (I);
ii) a second component being an additional active agent.

### BRIEF DESCRIPTION OF THE DRAWINGS

FIG. 1 shows the effects of acute feeding with corn oil+vehicle, corn oil+dipeptidyl peptidase 4 (DPP4) inhibitor or Compound B+DPP4 inhibitor, on area under the curve (AUC) (0-60 minutes) glucose stimulated active GLP-1 (pg/ml)×min in lean Sprague-Dawley (SPD) rats.

FIG. 2 shows the effects of corn oil+vehicle, corn oil+DPP4 inhibitor, compound A alone or Compound A+DPP4 inhibitor on active GLP-1 (pg/ml) at 24 h in lean SPD rats.

FIG. 3 show the effects of corn oil+vehicle, corn oil+DPP4 inhibitor, compound B alone or Compound B+DPP4 inhibitor on active GLP-1 (pg/ml) at 24 h in lean SPD rats.

FIG. 4 shows the effects of corn oil+vehicle, corn oil+DPP4 inhibitor, Compound A alone or Compound A+DPP4 inhibitor on plasma insulin (pg/ml) at 24 h in lean SPD rats.

FIG. 5 shows the effects of corn oil+vehicle, corn oil+DPP4 inhibitor, compound B alone or Compound B+DPP4 inhibitor on plasma insulin (pg/ml) at 24 h in lean SPD rats.

FIG. 6A shows the effects of a 28-day treatment with Compound B at 2 doses versus pioglitazone on glucose tolerance (0-120 mins) in a T2DM rodent model. FIG. 6B shows the effects of a 21-day treatment with Compound A versus pioglitazone on glucose tolerance in a T2DM rodent model.

FIG. 7 shows the effect on body weight of treatment with Compound B at 2 doses compared to no treatment in a dextran sodium sulfate (DSS)-induced colitis mouse model.

FIG. 8 shows the effect on colon length of treatment with Compound B at 2 doses (L, lower dose; H, higher dose) compared to no treatment (vehicle only) in a DSS-induced colitis mouse model.

FIG. 9 shows the survival rate of mice treated with Compound B at 2 different doses compared to no treatment in a DSS-induced colitis mouse model.

FIG. 10 shows the histological score of intestinal cross sections of mice treated with Compound B at 2 different doses compared to untreated mice in a DSS-induced colitis mouse model.

FIG. 11 shows the histological cross sections of mouse intestine from DSS-induced colitis mice that are untreated (FIGS. 11A-B), treated with a low dose (FIGS. 11C-D) or high dose of Compound B (FIGS. 11E-F) as compared to that of a mouse that was not induced with DSS (FIG. 11G). The scale bars for FIGS. 11A, C and E are 200 μm. The scale bars for FIGS. 11 B, D, F, and G are 50 μm.

FIG. 12 shows the effect of Compound B treatment on the relative colonic mRNA levels of a panel of cytokines and biomarkers associated with IBDs. The panel of genes tested include IL6 (FIG. 12A), IL1b (FIG. 12B), S100A8 (FIG. 12C), TNFα (FIG. 12D), Reg3g (FIG. 12E), and IL17a (FIG. 12F).

### DETAILED DESCRIPTION

The disclosed compositions and methods may be understood more readily by reference to the following detailed description taken in connection with the accompanying figures, which form a part of this disclosure. All references cited herein are incorporated by reference for any purpose. Where a reference and the specification conflict, the specification will control.

Disclosed herein are compounds that may stimulate enteroendocrine GLP-1 production. Also disclosed herein are compounds that reduce basal and/or postprandial hyperglycemia and/or increase postprandial plasma insulin concentrations. Further disclosed herein are compounds that treat and/or alleviate the symptoms of inflammatory bowel diseases (IBDs), such as intestinal inflammation, and induce remission IBD, maintain remission of IBD, reduce weight loss in subjects experiencing IBD symptoms, reduce decrease in colon length in subjects with IBD, reduce intestinal inflammation in subjects with IBD, and/or reduce intestinal injury in subjects with IBD. The compounds are unsaturated fatty acids structurally modified to comprise substituents in the α-position and preferably a heteroatom incorporated in the β-position. The compounds may be used either alone or in combination with one or more additional therapeutic agents.

Particular aspects of the disclosure are described in greater detail below. The terms and definitions as used in the present application and as clarified herein are intended to represent the meaning within the present disclosure.

The singular forms “a,” “an,” and “the” include plural reference unless the context dictates otherwise.

The terms “approximately” and “about” mean to be nearly the same as a referenced number or value. As used herein, the terms “approximately” and “about” should be generally understood to encompass ±5% of a specified amount, frequency, or value.

The terms “treat,” “treating,” and “treatment” include any therapeutic or prophylactic application that can benefit a human or non-human mammal. Both human and veterinary treatments are within the scope of the present disclosure. Treatment may be responsive to an existing condition or it may be prophylactic, i.e., preventative.

The terms “administer,” “administration,” and “administering” as used herein refer to (1) providing, giving, dosing and/or prescribing by either a health practitioner or his authorized agent or under his direction a compound or composition according to the present disclosure, and (2) putting into, taking or consuming by the human patient or person himself or herself, or non-human mammal a compound or composition according to the present disclosure.

The term “co-administration” or “coadministration” refers to administration of a (a) compound of Formula (I) or (II), or a pharmaceutically acceptable salt, solvate, or solvate of such a salt; and (b) an additional therapeutic agent, together in a coordinated fashion. For example, the co-administration can be simultaneous administration, sequential administration, overlapping administration, interval administration, continuous administration, or a combination thereof. The mode of administration may be different for the compounds and the additional agent(s), and the co-administration includes any mode of administration, such as oral, subcutaneous, sublingual, transmucosal, parenteral, intravenous, intra-arterial, intra-peritoneal, buccal, sublingual, topical, vaginal, rectal, ophthalmic, otic, nasal, inhaled, and transdermal, or a combination thereof. Examples of the parenteral administration include, but are not limited to intravenous (IV) administration, intraarterial administration, intramuscular administration, subcutaneous administration, intraosseous administration, intrathecal administration, or a combination thereof. The compound of formula (I) or (II) and the additional therapeutic agent can be independently administered, e.g. orally or parenterally. In one embodiment, the compound of Formula (I) or (II) is administered orally; and the additional therapeutic agent is administered parenterally. The parenteral administration may be conducted via injection or infusion. In another embodiment, both the compound of Formula (I), and the additional agent, such as a DPP-4 inhibitor, are administered orally.

The terms “preventing and/or treating” and “therapeutic and/or prophylactic treatment of” may interchangeably be used. Further, the terms “treatment” or “treating” may also encompass prophylactic treatment. Typically, the compounds of Formula (I) or Formula (II) will be used for treating, i.e. therapeutic treatment of, e.g. IBD; basal and/or postprandial hyperglycemia. However, the compounds of Formula (I) or Formula (II) may also be used for prophylactic treatment, e.g., of IBDs, including for maintenance of remission of IBDs. It is also foreseen that in some cases the compounds of Formula (I) or Formula (II) may be used as a potentiator of enteroendocrine GLP-1 secretion, promoting GSIS, satiety, slowing gastric emptying, inhibiting glucose-dependent glucagon secretion and reducing hepatic glucose production via GLP-1.

The term “pharmaceutically effective amount” means an amount sufficient to achieve the desired pharmacological and/or therapeutic effects, i.e., an amount of the disclosed compound and agents that are effective for the intended purpose. While individual subject/patient needs may vary, the determination of optimal ranges for effective amounts of the disclosed compound is within the skill of the art. Generally, the dosage regimen for treating a disease and/or condition with the compounds presently disclosed may be determined according to a variety of factors such as the type, age, weight, sex, diet, and/or medical condition of the subject/patient. The term “pharmaceutical composition” means a compound according to the present disclosure in any form suitable for medical use.

Compounds of the Disclosure

The compounds of Formula (I) and (II) may exist in various stereoisomeric forms, including enantiomers, diastereomers, or mixtures thereof. It will be understood that the invention encompasses all optical isomers of the compounds of Formula (I) and (II) as well as mixtures thereof. Hence, compounds of Formula (I) and (II) that exist as diastereomers, racemates, and/or enantiomers are within the scope of the present disclosure.

In one aspect, the invention provides a compound of Formula (I) for use in increasing GLP-1 production in a subject, wherein said compound is administered to the subject optionally in combination with one or more additional active agents.

In some embodiments, the invention provides a compound of Formula (I) for use in reducing basal or postprandial hyperglycemia and/or increasing postprandial plasma insulin concentrations in a subject, wherein said compound is administered to the subject optionally in combination with one or more additional active agents.

In some embodiments, the invention provides a compound of Formula (I) for use in treating IBD in a subject, inducing remission of IBD, maintaining remission of IBD, reducing weight loss in subjects experiencing IBD symptoms, reducing decrease in colon length, reducing intestinal inflammation in subjects with IBD, and/or reducing intestinal injury in subjects with IBD, wherein said compound is administered to the subject optionally in combination with one or more additional active agents.

The compounds of Formula (I) are:

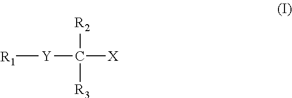

wherein R1 is selected from a C10-C22 alkenyl having 3-6 double bonds;
R2 and R3 are the same or different and are selected from a group of substituents consisting of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group, provided that R2 and R3 can be connected in order to form a cycloalkane like cyclopropane, cyclobutane, cyclopentane or cyclohexane, and provided that both R2 and R3 are not hydrogen;
X is a carboxylic acid or a derivative thereof, wherein the derivative is a carboxylate, such as a carboxylic ester; a glyceride; an anhydride; a carboxamide; a phospholipid; or a hydroxymethyl; or a prodrug thereof; and
Y is oxygen, sulphur, sulfoxide, sulfone or CH₂;
or a pharmaceutically acceptable salt, solvate, or solvate of such a salt.

In at least one embodiment, said compound is co-administered with one or more additional active agents. The subject is an animal, typically a mammal, and preferably a human being.

In some embodiments, Y is oxygen. In some embodiments, Y is sulphur.

Further, the compounds disclosed are for use in therapeutic treatment of hyperglycemia, such as for treatment of basal and/or postprandial hyperglycemia. In some embodiments, this may be through an increase in GSIS and/or a decrease in hepatic glucose output.

In at least one embodiment, R1 is a C18-C22 alkenyl having 3-6 double bonds, such as 5 or 6 double bonds, and preferably wherein one double bond is in the omega-3 position. In some embodiments, R1 is a C18-C22 alkenyl having 5 or 6 methylene interrupted double bonds, wherein the first double bond is between the 3^{rd} and 4th carbons from the omega end.

The α-substituents R2 and R3 are more preferably independently chosen from a hydrogen atom and linear, branched, and/or cyclic C1-C6 alkyl groups, with the proviso that both R2 and R3 cannot be hydrogen atoms. In one embodiment, at least one of R2 and R3 is a hydrogen atom, a methyl group, an ethyl group, a n-propyl group, and an isopropyl group, a butyl group or a pentyl group. In one embodiment, both R2 and R3 are a methyl group, an ethyl group or a n-propyl group, and most preferably both R2 and R3 are ethyl groups. In another embodiment, one of R2 and R3 is a hydrogen group and the other R2 or R3 is a C1-C3 alkyl group.

X preferably represents a carboxylic acid or a carboxylic ester; or a pharmaceutically acceptable salt, solvate, solvate of such a salt. More preferably, X is a carboxylic acid group providing the modified fatty acid in the free acid form.

Y is preferably oxygen, sulphur, sulfoxide or sulfone, and is most preferably oxygen or sulphur.

More preferable for compounds of Formula (I),
R2 and R3 are independently chosen from a hydrogen atom or linear, branched, and/or cyclic C1-C6 alkyl groups, with the proviso that R2 and R3 cannot both be hydrogen atoms;
X is a carboxylic acid or a carboxylic ester; or a pharmaceutically acceptable salt, solvate, or solvate of such a salt; and
Y is oxygen or sulphur.

In some embodiments, for compounds of Formula (I),
R2 and R3 are independently chosen from a hydrogen atom or linear, branched, and/or cyclic C1-C6 alkyl groups, with the proviso that R2 and R3 cannot both be hydrogen atoms;
X is a carboxylic acid or a carboxylic ester; or a pharmaceutically acceptable salt, solvate, or solvate of such a salt; and
Y is sulphur.

In some embodiments, for compounds of Formula (I),
R2 and R3 are independently chosen from a hydrogen atom or linear, branched, and/or cyclic C1-C6 alkyl groups, with the proviso that R2 and R3 cannot both be hydrogen atoms; and
X is a carboxylic acid or a carboxylic ester; or a pharmaceutically acceptable salt, solvate, or solvate of such a salt; and
Y is oxygen.

In at least one embodiment, R1 is a C20 alkenyl group having 5 methylene interrupted double bond such that the first double bond is in the omega-3 position (i.e., a C20:5n3 chain), and more preferably the compound of Formula (I) for use, is a compound of Formula (II):

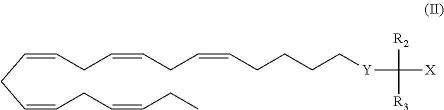

wherein R2, R3, Y and X are defined as for Formula (I),

for use in increasing GLP-1 production, reducing basal and/or postprandial hyperglycemia, reducing postprandial plasma insulin levels, treating IBD in a subject, reducing intestinal inflammation in a subject with IBD, inducing remission of IBD, maintaining remission of IBD, reducing weight loss in subjects experiencing IBD symptoms, reducing decrease in colon length in a subject with IBD, reducing intestinal inflammation in a subject with IBD, and/or reducing intestinal injury in subjects with IBD.

Formula (II) hence represents a limited group of the compounds of Formula (I).

More preferable for the compounds of Formula (II),
R2 and R3 are independently chosen from a hydrogen atom or linear, branched, and/or cyclic C1-C6 alkyl groups, with the proviso that both R2 and R3 cannot be hydrogen atoms;
X is a carboxylic acid or a carboxylic ester; or a pharmaceutically acceptable salt, solvate, or solvate of such a salt; and
Y is oxygen or sulphur.

In some embodiments, for the compounds of Formula (II),
R2 and R3 are independently chosen from a hydrogen atom or linear, branched, and/or cyclic C1-C6 alkyl groups, with the proviso that R2 and R3 cannot both be hydrogen atoms;
X is a carboxylic acid or a carboxylic ester; or a pharmaceutically acceptable salt, solvate, or solvate of such a salt; and
Y is sulphur.

In some embodiments, for the compounds of Formula (II),
R2 and R3 are independently chosen from a hydrogen atom or linear, branched, and/or cyclic C1-C6 alkyl groups, with the proviso that R2 and R3 cannot both be hydrogen atoms;
X is a carboxylic acid or a carboxylic ester; or a pharmaceutically acceptable salt, solvate, or solvate of such a salt; and
Y is oxygen.

In cases in which R2 and R3 are different, the compounds of Formula (I) and Formula (II) are capable of existing in stereoisomeric forms. It will be understood that the invention encompasses all optical isomers of the compounds of Formula (I) and Formula (II) and mixtures thereof.

For compounds of both Formula (I) and Formula (II), in at least one embodiment, R2 and R3 are independently selected from the group of a hydrogen atom, a methyl group, an ethyl group, a n-propyl group, an isopropyl group, a butyl group and a pentyl group. In some embodiments, R2 and R3 cannot both be a hydrogen atom. In at least one embodiment, R2 and R3 are independently selected from the group of a hydrogen atom, a methyl group, and an ethyl group. In some embodiments, R2 and R3 are independently selected from the group of a hydrogen atom, a methyl group, and an ethyl group, with the proviso that R2 and R3 cannot both be a hydrogen atom.

In at least one embodiment, one of R2 and R3 is a hydrogen atom and the other one of R2 and R3 is chosen from a C1-C3 alkyl group. In one embodiment, one of R2 and R3 is a hydrogen atom and the other one of R2 and R3 is selected from the group of a methyl group and an ethyl group, and most preferably, one of R2 and R3 is a hydrogen atom and the other one is an ethyl group.

In another embodiment, both R2 and R3 are C1-C3 alkyl groups. In one embodiment R2 and R3 are the same or different and each are independently chosen from a methyl group, an ethyl group, an n-propyl group, or an isopropyl group. In a preferred embodiment both R2 and R3 are the same and are selected from a pair of methyl groups, a pair of ethyl groups, a pair of n-propyl groups or a pair of isopropyl groups. In at least one preferred embodiment R2 and R3 are ethyl groups. In one embodiment, one of R2 and R3 is a methyl group and the other one is an ethyl group. In one embodiment, one of R2 and R3 is an ethyl group and the other one is an n-propyl group.

In at least one embodiment, the compound is present in its various stereoisomeric forms, such as an enantiomer (R or S), a diastereomer, or mixtures thereof. In at least one embodiment, the compound is present in racemic form. Particularly, in those cases, were R2 and R3 are different, the compounds of Formula (I) and Formula (II) are capable of existing in stereoisomeric forms. It will be understood that the invention encompasses all optical isomers of the compounds of Formula (I) and Formula (II) and mixtures thereof.

In cases in which the compound according to Formula (I) is a salt of a counter-ion with at least one stereogenic center, or ester of an alcohol with at least one stereogenic center, the compound may have multiple stereocenters. In those situations, the compounds of the present disclosure may exist as diastereomers. Thus, in at least one embodiment, the compounds of the present disclosure are present as at least one diastereomer.

In at least one embodiment, when Y is oxygen, R2 and R3 are preferably different, and more preferably one of R2 and R3 is ethyl and the other is hydrogen. In other embodiments, when Y is sulphur, R2 and R3 are preferably the same, and more preferably both R2 and R3 are ethyl.

In at least one embodiment, the compound for use of the present disclosure is 2-(((5Z,8Z,11Z,14Z,17Z)-icosa-5,8,11,14,17-pentaen-1-yl)oxy)butanoic acid (Compound A):

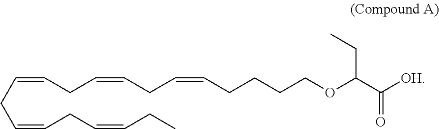 [SMILES: CC/C=C\C/C=C\C/C=C\C/C=C\C/C=C\CCCCOC(CC)C(=O)O]

In at least one embodiment, the compound for use of the present disclosure is Compound A present in its S and/or R form represented by the formulas:

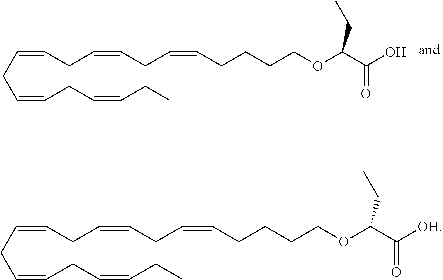 [SMILES: CC/C=C\C/C=C\C/C=C\C/C=C\C/C=C\CCCCO[C@@H](CC)C(=O)O ; CC/C=C\C/C=C\C/C=C\C/C=C\C/C=C\CCCCO[C@H](CC)C(=O)O]

In at least one embodiment, the compound for use of the present disclosure is 2-ethyl-2-((5Z,8Z,11 Z,14Z,17Z)-icosa-5,8,11,14,17-pentaenylthio)butanoic acid (Compound B):

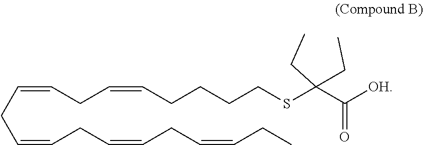 [SMILES: CC/C=C\C/C=C\C/C=C\C/C=C\C/C=C\CCCCSC(CC)(CC)C(=O)O]

In a further aspect, the invention provides a combination product comprising a first and a second component, wherein the first component is a compound of Formula (I):

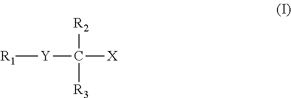

wherein R1 is selected from a C10-C22 alkenyl having 3-6 double bonds;
R2 and R3 are the same or different and are selected from a group of substituents consisting of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group, provided that R2 and R3 can be connected in order to form a cycloalkane like cyclopropane, cyclobutane, cyclopentane or cyclohexane, and provided that both R2 and R3 are not hydrogen;
X is a carboxylic acid or a derivative thereof, wherein the derivative is a carboxylate, such as a carboxylic ester; a glyceride; an anhydride; a carboxamide; a phospholipid; or a hydroxymethyl; or a prodrug thereof;
Y is oxygen, sulphur, sulfoxide, sulfone and CH₂;
or a pharmaceutically acceptable salt, solvate, or solvate of such a salt; and
the second component is an additional active agent.

The embodiments and features described in the context of the first aspects directed to the method and use, also apply to this other aspect of the invention. Hence, the first component of the combined product is selected from the group of compounds disclosed in the first aspect directed to the compounds for use. In a preferred aspect, the combined product comprise a compound of Formula (II), as the first component. In one embodiment, the combined product comprises compound B as the first component. In another embodiment, the combined product comprises compound A, as the first component.

The first component of the combined product, i.e. the compound of Formula (I) or (II) may be administered as a medicament, such as in a pharmaceutical composition. The composition presently disclosed may comprise at least one compound as disclosed and optionally at least one non-active pharmaceutical ingredient, i.e., excipient. Non-active ingredients may solubilize, suspend, thicken, dilute, emulsify, stabilize, preserve, protect, color, flavor, and/or fashion active ingredients into an applicable and efficacious preparation, such that it may be safe, convenient, and/or otherwise acceptable for use. Examples of excipients include, but are not limited to, solvents, carriers, diluents, binders, fillers, sweeteners, aromas, pH modifiers, viscosity modifiers, antioxidants, extenders, humectants, disintegrating agents, solution-retarding agents, absorption accelerators, wetting agents, absorbents, lubricants, coloring agents, dispersing agents, and preservatives. Excipients may have more than one role or function, or may be classified in more than one group; classifications are descriptive only and are not intended to be limiting. In some embodiments, for example, the at least one excipient may be chosen from corn starch, lactose, glucose, microcrystalline cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose, and fatty substances such as hard fat or suitable mixtures thereof.

In some embodiments, the composition comprise at least one compound of Formula (I), such as one of Formula (II), and at least one pharmaceutically acceptable antioxidant, e.g., tocopherol such as a/pha-tocopherol, beta-tocopherol, gamma-tocopherol, and delta-tocopherol, or mixtures thereof, BHA such as 2-tert-butyl-4-hydroxyanisole and 3-tert-butyl-4-hydroxyanisole, or mixtures thereof and BHT (3,5-di-tert-butyl-4-hydroxytoluene), or mixtures thereof. The composition presently disclosed may be formulated in oral administration forms, e.g., tablets or gelatin soft or hard capsules. The dosage form can be of any shape suitable for oral administration, such as spherical, oval, ellipsoidal, cube-shaped, regular, and/or irregular shaped. The composition may be in the form of a gelatin capsule or a tablet.

The second component of the combined product, the additional active agent, is formulated as suitable for the type of agent it is, and depends on several factors, including the mode of administration of the agent. For example, several DPP-4 inhibitors that can be taken orally as tablets have been developed. In a preferred embodiment, both the first component and the second component are provided in forms for oral administration.

A suitable daily dosage of the compound of Formula (I), may range from about 5 mg to about 4 g, such as from about 5 mg to about 2 g. For example, in some embodiments, the daily dose ranges from about 10 mg to about 1.5 g, from about 50 mg to about 1 g, from about 100 mg to about 1 g, from about 150 mg to about 900 mg, from about 50 mg to about 800 mg, from about 100 mg to about 800 mg, from about 100 mg to about 600 mg, from about 150 to about 550 mg, or from about 200 to about 500 mg. In some embodiments, the daily dose ranges from about 200 mg to about 400 mg, from about 250 mg to about 350 mg, from about 300 to about 500 mg, from about 400 mg to about 600 mg, from about 550 mg to about 650 mg, or from about 600 mg to about 800 mg.

In some embodiments, the daily dose of a compound of Formula (I) ranges from about 900 mg to about 1.6 g. In some embodiments, the daily dose of a compound of Formula (I) ranges from about 1 g to about 1.5 g.

In some embodiments, the compound of formula (I) is administered in a daily dosage of 600 mg. In some embodiments, the compound of Formula (I) is administered at a daily dosage of 300 mg. In some embodiments, the compound of Formula (I) is administered at a daily dosage of 250 mg. Preferably, the compound of Formula (I) is administered at a daily dosage of 300 mg, 600 mg, 1 g, or 1.5 g per day.

In at least one embodiment, the daily dose ranges from about 200 mg to about 600 mg. In at least one embodiment, the daily dose is about 50 mg, about 100 mg, about 200 mg, about 300 mg, about 400 mg, about 500 mg, about 600 mg, about 700 mg, about 800 mg, or about 900 mg. In some embodiments, the daily dosage is 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, or 900 mg. The compound(s) may be administered, for example, once, twice, or three times per day. In at least one embodiment, the compound of Formula (I) is administered in an amount ranging from about 200 mg to about 800 mg per dose. In at least one embodiment, the compound of Formula (I) is administered once per day. The dose of the additional active agent depends on the type of agent selected, and should be in accordance with the approved amounts for the specific agent. Preferably, the compound of Formula (I) is administered once per day at a dosage of 300 mg or 600 mg.

In at least one embodiment, the daily dose ranges from about 900 mg to 1.6 g. In at least one embodiment, the daily dose is about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, or about 1600 mg.

In at least one embodiment, the compound of Formula (II) is administered in an amount ranging from about 200 mg to about 800 mg or in amount ranging from about 900 mg to about 1.6 g per dose. In at least one embodiment, the compound of Formula (II) is administered once per day. In some embodiments, the compound of Formula (II) is administered once per day at a dose of 1.5 g. In some embodiments, the compound of Formula (II) is administered once per day at a dose of 1.25 g. In some embodiments, the compound of Formula (II) is administered once per day at a dose of 1 g. In at least one embodiment, the compound of Formula (II) is administered once per day at a dose of 750 mg. In some embodiments, the compound of Formula (II) is administered once per day at a dose of 600 mg. In some embodiments, the compound of Formula (II) is administered once per day at a dose of 500 mg. In some embodiments, the compound of Formula (II) is administered once per day at a dose of 300 mg. In some embodiments, the compound of Formula (II) is administered once per day at a dose of 250 mg. Preferably, the compound of Formula (II) is administered once per day at a dose of 300 mg, 600 mg, 1 g, or 1.5 g.

Preferably, Compound A is administered once per day at a dose of 300 mg or 600 mg. Preferably, Compound B is administered once per day at a dose ranging from 1 g to 1.5 g.

Compounds of Formula (I) and Formula (II) can be prepared as described, for example, in PCT Applications WO 2009/061208, WO 2010/008299, WO2010/128401, WO 2011/089529, WO 2016/156912 and according to Examples below. In addition, Compound A can be prepared as described, for example, in PCT Application WO2014/132135. Compound B can be prepared as described, for example, in WO 2010/008299.

Increasing GLP-1

It has now been found that the disclosed structurally modified fatty acids have an improved ability to increase GLP-1 concentrations versus unmodified long-chain fatty acids. Hence, more particularly, the disclosure provides compounds for use as potentiators of GSIS and as inhibitors of hepatic glucose output.

It should be noted that embodiments and features described in the context of one aspect of the present disclosure also apply to the other aspects of the invention. Particularly, the embodiments applying to the method of increasing GLP-1 according to the present disclosure also apply to the aspect directed to a compound for use, or a composition comprising the compound co-administered with another drug for the use, such as in increasing GLP-1, all according to the present disclosure.

It has now been found that specific structurally modified fatty acids according to Formula I, or more preferably as specified by Formula II, have an improved ability to stimulate enteroendocrine GLP-1 secretion. Without being bound by theory, the structurally modified fatty acids may achieve this effect by:
a) having reduced systemic absorption and thereby targeting enteroendocrine L-cells in the distal small intestine and large intestine; and/or
b) having prolonged contact with enteroendocrine L-cells and thereby achieving an extended release of GLP-1 from the gut; and/or
c) resisting incorporation into chylomicrons and thereby facilitating greater free fatty acid delivery to enteroendocrine L-cells on the vascular side of the gut wall/embedded in the gut lining; and/or
d) resisting intracellular esterification into complex lipids and thereby increasing substrate availability for CYP450/lipoxygenase modification for the generation of more potent ligands for autocrine GPR40/GPR120 binding; and/or
e) inhibiting hepatic/intestinal DPP-4 activity and thereby decreasing GLP-1 degradation.

Improving Glycemic Control

The compounds for use further provide a means for increasing GSIS, promoting satiety, slowing gastric emptying, inhibiting glucose-dependent glucacon secretion, and/or reducing hepatic glucose production.

In further embodiments, the compounds are for use in therapeutic treatment of elevated blood glucose levels. More specifically, the invention provides compounds of Formula (I) for the use in treatment of basal and/or postprandial hyperglycemia. Without being bound by theory, this is possibly due to an increase in postprandial and basal GLP-1 and GSIS and/or decreasing hepatic glucose output.

In some embodiments, the compounds are for use in improving glycemic control, such as reducing basal and/or postprandial hyperglycemia, and/or increasing postprandial plasma insulin concentrations. In some embodiments, the compounds are for use in reducing basal plasma insulin concentrations. In some embodiments, the compounds are for use in reducing blood HbA1c and/or reducing HOMA-IR. In some embodiments, the compounds are for use in reducing plasma ALT in subjects with T2DM. In preferred embodiments, the compounds are for use in reducing postprandial hyperglycemia and/or increasing postprandial plasma insulin concentrations.

Glycemic control is the regulation of plasma glucose levels. Improving glycemic control can be achieved by reducing plasma glucose levels, by increasing postprandial plasma insulin levels and/or by increasing cellular insulin sensitivity, and/or by reducing hepatic glucose output.

The term “reducing basal hyperglycemia” in a subject administered a compound of Formula (I) indicates that basal hyperglycemia is reduced compared with a subject that is not administered a compound of Formula (I). Basal hyperglycemia in humans is defined as plasma glucose levels of 130 mg/dl and above 8 hours after eating. The term “reducing postprandial hyperglycemia” in a subject administered a compound of Formula (I) indicates that postprandial hyperglycemia is reduced compared with a subject that is not administered a compound of Formula (I). Postprandial hyperglycemia in humans is defined as plasma glucose levels of 180 mg/dl and above 1-2 hours after eating. For both terms, a reduction in hyperglycemia represents a reduction in plasma or blood glucose levels.

The term “increasing postprandial plasma insulin concentrations” in a subject administered a compound of Formula (I) indicates that the plasma insulin concentration of the subject is increased postprandial compared to a subject that is not administered a compound of Formula (I). The term “decreasing basal plasma insulin concentrations” in a subject administered a compound of Formula (I) indicates that the basal plasma insulin concentration of the subject is decreased compared to a subject that is not administered a compound of Formula (I). The term “plasma insulin concentration” is interchangeable with the term “plasma insulin level.”

The term “decreasing HbA1c levels” in a subject administered a compound of Formula (I) indicates that the level of HbA1c of the subject is decreased compared to a subject that is not administered a compound of Formula (I). The term “decreasing plasma ALT levels” in a subject with T2DM administered a compound of Formula (I) indicates that the plasma ALT level of the subject is decreased compared to a subject with T2DM that is not administered a compound of Formula (I).

The term “decreasing HOMA-IR” in a subject administered a compound of Formula (I) indicates that the HOMA-IR calculation for the subject is decreased compared to a subject that is not administered a compound of Formula (I). HOMA-IR is an assessment of insulin resistance and can be calculated by the following formula: fasting insulin (micro U/L)×fasting glucose (nmol/L)/22.5.

As provided in Biological Example 1, a compound of Formula (I) increased active GLP-1 concentrations in lean SPD rats during the first 60 minutes after an oral glucose load compared with rats that were not administered a compound of Formula (I). As described above, GLP-1 increases glucose stimulated insulin secretion (GSIS), which results in increased postprandial plasma insulin levels. Biological Examples 2-5 show that lean SPD rats administered compounds of Formula (I) have both increased GLP-1 levels and increased plasma insulin levels 24 hours after an oral glucose load compared with rats that were not administered a compound of Formula (I). These data support that plasma insulin concentration is likewise increased during the first 60 minutes after an oral glucose load in rats administered a compound of Formula (I).

As provided in Biological Examples 4 and 5, compounds of Formula (I) increase plasma insulin levels in lean SPD rats 24 hours after an oral glucose load compared with rats that were not administered a compound of Formula (I). In some embodiments, the compounds of Formula (I) increase plasma insulin levels by 25% compared to subjects not administered a compound of Formula (I). In some embodiments, the compounds of Formula (I) increase plasma insulin levels by 25% compared to subjects administered a DPP4 inhibitor but not a compound of Formula (I). In some embodiments, the compounds of Formula (I) are administered with a DDP4 inhibitor and increase plasma insulin levels by 40% compared to subjects not administered a compound of Formula (I). In some embodiments, the compounds of Formula (I) result in increased plasma insulin levels 24 hours after an oral glucose load.

As provided in Biological Examples 6 and 14, compounds of Formula (I) decrease postprandial glucose levels in a mouse model of T2DM compared with mice that were not administered a compound of Formula (I). In some embodiments, the compounds of Formula (I) decrease plasma glucose levels by 25% 15 minutes and 30 minutes postprandial in subjects with T2DM compared with subjects with T2DM not administered a compound of Formula (I). In some embodiments, the compounds of Formula (I) decrease plasma glucose levels by 50% 15 minutes and 30 minutes postprandial in subjects with T2DM compared with subjects with T2DM not administered a compound of Formula (I). In some embodiments, the compounds of Formula (I) decrease plasma glucose levels 15 minutes and 30 minutes postprandial in subjects with T2DM compared with subjects with T2DM who are administered pioglitazone but not a compound of Formula (I). In some embodiments, the compounds of Formula (I) decrease plasma glucose levels from 15 minutes to 90 minutes postprandial in subjects with T2DM compared with subjects with T2DM not administered a compound of Formula (I). In some embodiments, the compounds of Formula (I) decrease plasma glucose levels by 50% 60 minutes postprandial in subjects with T2DM compared with subjects with T2DM not administered a compound of Formula (I).

As described in Biological Example 14, chronic treatment with a compound of Formula (I) decreases basal glucose levels in a mouse model of T2DM compared with mice that were not administered a compound of Formula (I). In some embodiments, the compounds of Formula (I) decrease basal plasma glucose levels in subjects with T2DM compared with subjects with T2DM not administered a compound of Formula (I). In some embodiments, the compounds of Formula (I) decrease basal plasma glucose levels by 25% in subjects with T2DM compared with subjects with T2DM not administered a compound of Formula (I). In some embodiments, the compounds of Formula (I) decrease basal plasma glucose levels by 30% in subjects with T2DM compared with subjects with T2DM not administered a compound of Formula (I). In some embodiments, the compounds of Formula (I) decrease basal plasma glucose levels by 35% in subjects with T2DM compared with subjects with T2DM not administered a compound of Formula (I). In some embodiments, the compounds of Formula (I) decrease basal plasma glucose levels by 40% in subjects with T2DM compared with subjects with T2DM not administered a compound of Formula (I). In some embodiments, the compounds of Formula (I) decrease basal plasma glucose levels by 45% in subjects with T2DM compared with subjects with T2DM not administered a compound of Formula (I). In some embodiments, the compounds of Formula (I) decrease basal plasma glucose levels by 50% in subjects with T2DM compared with subjects with T2DM not administered a compound of Formula (I).

As described in Biological Example 14, chronic treatment with a compound of Formula (I) decreases basal plasma insulin levels in a mouse model of T2DM compared with mice that were not administered a compound of Formula (I). In some embodiments, the compounds of Formula (I) decrease basal plasma insulin levels in subjects with T2DM compared with subjects with T2DM not administered a compound of Formula (I). In some embodiments, the compounds of Formula (I) decrease basal plasma insulin levels by 50% in subjects with T2DM compared with subjects with T2DM not administered a compound of Formula (I). In some embodiments, the compounds of Formula (I) decrease basal plasma insulin levels by 60% in subjects with T2DM compared with subjects with T2DM not administered a compound of Formula (I). In some embodiments, the compounds of Formula (I) decrease basal plasma insulin levels by 70% in subjects with T2DM compared with subjects with T2DM not administered a compound of Formula (I).

As described in Biological Example 14, chronic treatment with a compound of Formula (I) decreases HBA1c levels in a mouse model of T2DM compared with mice that were not administered a compound of Formula (I). In some embodiments, the compounds of Formula (I) decrease HBA1c levels in subjects with T2DM compared with subjects with T2DM not administered a compound of Formula (I). In some embodiments, the compounds of Formula (I) decrease HBA1c levels by 25% in subjects with T2DM compared with subjects with T2DM not administered a compound of Formula (I). In some embodiments, the compounds of Formula (I) decrease HBA1c levels by 30% in subjects with T2DM compared with subjects with T2DM not administered a compound of Formula (I). In some embodiments, the compounds of Formula (I) decrease HBA1c levels by 40% in subjects with T2DM compared with subjects with T2DM not administered a compound of Formula (I).

As described in Biological Example 14, chronic treatment with a compound of Formula (I) decreases HOMA-IR values in a mouse model of T2DM compared with mice that were not administered a compound of Formula (I). In some embodiments, the compounds of Formula (I) decrease HOMA-IR value in subjects with T2DM compared with subjects with T2DM not administered a compound of Formula (I). In some embodiments, the compounds of Formula (I) decrease HOMA-IR value by 50% in subjects with T2DM compared with subjects with T2DM not administered a compound of Formula (I). In some embodiments, the compounds of Formula (I) decrease HOMA-IR value by 60% in subjects with T2DM compared with subjects with T2DM not administered a compound of Formula (I). In some embodiments, the compounds of Formula (I) decrease HOMA-IR value by 70% in subjects with T2DM compared with subjects with T2DM not administered a compound of Formula (I). In some embodiments, the compounds of Formula (I) decrease HOMA-IR value by 80% in subjects with T2DM compared with subjects with T2DM not administered a compound of Formula (I).

As described in Biological Example 14, chronic treatment with a compound of Formula (I) decreases plasma alanine aminotransferase (ALT) levels in a mouse model of T2DM compared with mice that were not administered a compound of Formula (I). In some embodiments, the compounds of Formula (I) decrease plasma ALT levels in subjects with T2DM compared with subjects with T2DM not administered a compound of Formula (I). In some embodiments, the compounds of Formula (I) decrease plasma ALT levels by 20% in subjects with T2DM compared with subjects with T2DM not administered a compound of Formula (I). In some embodiments, the compounds of Formula (I) decrease plasma ALT levels by 25% in subjects with T2DM compared with subjects with T2DM not administered a compound of Formula (I). In some embodiments, the compounds of Formula (I) decrease plasma ALT levels by 30% in subjects with T2DM compared with subjects with T2DM not administered a compound of Formula (I).

The disclosed compounds are also suitable for use for the manufacture of a medicament for the described indications. For example, the disclosure provides for use of the compounds of Formula (I) for the manufacture of a medicament for reducing basal and/or postprandial hyperglycemia and increasing postprandial plasma insulin levels.

In one embodiment, the method and compounds for use of the invention, provides use of at least two different active agents, the compound of Formula (I) or (II), and an additional active agent, preferably a DPP-4 inhibitor, respectively. The at least two active agents can be seen as a “Combined product”, wherein the agents are e.g. separately packed and wherein both agents are required to achieve the optimal intended effect. According to the invention, the compound of Formula (I) or (II) is hence co-administered with an additional active agent. In some embodiments, the additional active agent is a dipeptidyl peptidase-4 (DPP-4) inhibitor and this agent and the compound of Formula (I) have synergistic effect on increasing plasma GLP-1 concentrations. A non-limiting example list of dipeptidyl peptidase inhibitors include: Sitagliptin, Vildagliptin, Saxagliptin, Linagliptin, Gemigliptin, Anagliptin, Teneligliptin, Alogliptin, Trelagliptin, Omarigliptin, Evogliptin, Dutogliptin. Hence, the method and use as disclosed include optional administration of any of these or similar DPP-4 inhibitors.

A series of experiments have been performed to assess both the effects of specific structural modifications to long chain fatty acids on gut retention versus systemic absorption in addition to effects upon acute (0-60 mins) and prolonged (24 h) plasma GLP-1 and insulin concentration in rodents.

As provided in the Examples, the studies support the notion that combining a DPP-4 inhibitor with an unsaturated fatty acid with substituents in the α-position, i.e. a compound of Formula (I) or (II), such as Compound B, is superior to either treatment alone for increasing plasma GLP-1 concentrations. As both postprandial and elevated basal hyperglycemia can be reduced via potentiation of glucose stimulated insulin secretion and/or decreased hepatic glucose output, these findings demonstrate superiority of structurally modified fatty acids (e.g. Compound A or B) in combination with a DPP-4 inhibitor versus a DPP-4 inhibitor alone. Overall, the data suggest that a combination of a DPP-4 inhibitor with an oxygen/sulphur containing structurally modified fatty acid may achieve a synergistic effect for both increasing postprandial and basal GLP-1 and insulin concentrations.

Despite the widespread use of oral DPP-4 inhibitors as efficacious type 2 diabetes (T2DM) drugs, their ability to increase plasma GLP-1 concentrations is ultimately dependent on endogenous GLP-1 production. Endogenous GLP-1 occurs primarily after food intake and diminishes in the late postprandial period and during overnight fasting as food derived intestinal GPR40/120 ligands are absorbed from the upper GI tract. DPP-4 inhibitors increase the half-life of GLP-1 from several minutes to 2-4 hours. The ability to harness the GPR40/120 rich enteroendocrine cells in the lower gut would thus be highly desirable, both to increase total GLP-1 production and to provide prolonged GLP-1 production from the gut in the fasting state. Thus, the novel and remarkable increases in active GLP-1 achieved with Compound B, not only in response to an acute glucose load (0-60 mins GLP-1) but also at 24 hours (when the DPP-4 inhibitor no longer increased GLP-1 levels with corn oil), suggests that Compound B is able to induce GLP-1 production from both the upper and the lower gut, thereby providing prolonged elevated GLP-1 levels. In combination with the elevated insulin levels at 24 h, this suggests Compound A or B could be used either alone or preferably with a DPP-4 inhibitor to increase both acute and chronic GLP-1 and thereby reduce both postprandial and basal plasma glucose.

As the major determinant of glycated haemoglobin in badly controlled diabetics is basal and not postprandial glucose, this prolonged effect on plasma GLP-1 could be of considerable benefit in the prophylactic treatment of macro- and microvascular complications associated with prolonged elevated glucose. Remarkably, the acute effects were achieved at a fraction of the dose (75 mg/kg) typically used as an oral bolus of fat needed to induce GLP-1 production. These effects are particularly surprising in relation to previous studies (Morishita M et al., J. Control. Release, 2008, 132(2):99-104) showing that naturally occurring long-chain omega-3 fatty acids had no effect on GLP-1 when administered via the stomach and jejunum. This suggests that the effects of Compound B on GLP-1 are not only related to its ability to reach the lower GI tract. Overall the data support the use of structurally modified fatty acids according to Formula (I) or (II) as activators of enteroendocrine GLP-1 production, which can be optimally combined with DPP-4 inhibitors, for use as a potentiator of glucose stimulated and/or basal insulin production, promoting satiety, slowing gastric emptying, inhibiting glucose-dependent glucagon secretion and reducing hepatic glucose production via GLP-1.

Based on the above findings, the compounds of Formula (I), or preferably of Formula (II), may be optimally co-administered with a DPP-4 inhibitor. Further compounds may be administered to therapeutically and/or prophylactically treat a condition where activation of enteroendocrine GPR40/GPR120 is desirable.

The Examples highlight the potential of the structurally modified fatty acids with substituents in the α-position to be combined with a DPP-4 inhibitor. These combinations may not only improve efficacy related outcomes versus monotherapy, but may also improve safety, tolerability and compliance versus an injectable GLP-1 agonist as both the DPP-4 inhibitor and Compound A and B can be administered orally, thereby negating risk of injection site reactions. As both Compound A and B have been demonstrated to significantly reduce atherogenic lipids in humans (Compound A) and APOE*3.CETP mice (Compounds A and B) a combination of either of Compound A or B with a DPP-4 inhibitor could optimise both plasma GLP-1 concentrations and treat any associated dyslipidemia. This may be advantageous given the known association of insulin resistance/T2DM and hyperlipidemia with increased morbidity and mortality.

In some embodiments, the compounds of Formula (I) will be used in conjunction with an additional active agent. In some embodiments, the additional active agent is preferably an inhibitor of the enzyme that inactivates incretins, hence the additional active agent is preferably a dipeptidyl peptidase-4 (DPP-4) inhibitor. Preferably, the DPP-4 inhibitor is selected from the non-limiting example list of Sitagliptin, Vildagliptin, Saxagliptin, Linagliptin, Gemigliptin, Anagliptin, Teneligliptin, Alogliptin, Trelagliptin, Omarigliptin, Evogliptin, Dutogliptin. In one embodiment, the first and second components have a synergistic effect on increasing plasma incretin concentrations, such as GLP-1.

Treating Inflammatory Bowel Diseases

The invention also provides compounds for use as a treatment for gastrointestinal disorders where activation of enteroendocrine GPR40/GPR120 and/or stimulation of GLP-1 is desirable. Such GLP-1 related disorders include inflammation in the gut, specifically in inflammatory bowel diseases, such as ulcerative colitis (UC), Crohn's disease, and indeterminate colitis.

It has now been found that structurally modified fatty acids according to Formula (I), or more preferably as specified by Formula II, may treat, or alleviate the symptoms of, inflammatory bowel disease (IBD). In one aspect, the compounds are for use in therapeutic treatment of IBD. IBD is a group of chronic, immune dysregulation disorders of the gut, and include but are not limited to Crohn's disease (CD), ulcerative colitis (UC) and indeterminate colitis. In some embodiments, the compounds disclosed herein are for use in the treatment of Crohn's disease. In some embodiments, the compounds disclosed herein are for use in the treatment of ulcerative colitis. In some embodiments, the compounds disclosed herein are for use in the treatment of indeterminate colitis. Further, the compounds are for use in therapeutic, symptomatic and/or prophylactic treatment of IBD.

In some embodiments, the compounds are for use in reducing intestinal inflammation associated with IBD. In some embodiments, the compounds are for use in inducing remission of IBD. In some embodiments, the compounds are for use in the maintenance of remission of IBD. In some embodiments, the compounds are for use in preventing weight loss in subjects experiencing IBD symptoms. In some embodiments, the compounds are for use in reducing a decrease in colon length in a subject with IBD. In some embodiments, the compounds are for use in reducing intestinal injury in a subject with IBD.

The term “reducing intestinal inflammation” in a subject with IBD administered a compound of Formula (I) indicates that intestinal inflammation is reduced compared with a subject with IBD that is not administered a compound of Formula (I). Intestinal inflammation can be assessed by histological scoring, such as is described in Biological Example 12, and by expression of inflammatory markers, such as is described in Biological Example 12. Intestinal inflammation can also be assessed by clinical as well as clinical-histological composite scores including endoscopic-histological features and clinical-laboratory parameters applicable to the 3 forms of IBD. de Jong et al., Clin Gastroenterol Hepatol., 2018, 16(5):648-663.

The term “inducing remission” in a subject with IBD administered a compound of Formula (I) indicates that remission from IBD symptoms and/or intestinal inflammation is induced compared with a subject with IBD that is not administered a compound of Formula (I). The term “remission” encompasses both periods during which symptoms are ameliorated or absent and periods during which intestinal inflammation is absent.

The term “maintenance of remission” in a subject with IBD administered a compound of Formula (I) indicates that remission of IBD symptoms and/or intestinal inflammation is maintained for a longer period compared with a subject with IBD that is not administered a compound of Formula (I).

The term “preventing weight loss” in a subject with IBD symptoms and administered a compound of Formula (I) indicates that weight loss is reduced compared with a subject with IBD symptoms that is not administered a compound of Formula (I). Preventing weight loss encompasses reducing the amount of weight that is lost and maintaining initial body weight.

The term “reducing a decrease in colon length” in a subject with IBD administered a compound of Formula (I) indicates that a decrease in colon length is reduced or ameliorated compared with a subject with IBD that is not administered a compound of Formula (I).

The term “intestinal injury” as used herein describes injury to the intestinal epithelial cells and/or mucosal surface. The term “reducing intestinal injury” in a subject with IBD administered a compound of Formula (I) indicates that intestinal epithelial and/or mucosal injury is reduced compared with a subject with IBD that is not administered a compound of Formula (I). Intestinal epithelial and mucosal injury can be assessed by histological scoring, such as is described in Biological Example 12. Other methods for assessing intestinal epithelial and mucosal injury include immunological profiling using, e.g., Immunohistochemistry, FACS analysis, PCR and proteomic/phosphoproteomic profiling of the intestinal mucosa, and using surrogate serum/plasma or fecal markers of intestinal and general inflammation due to IBD. Di Ruscio et al., Inflamm Bowel Dis., 2017, 24(1):78-92; Iborra et al., Gastrointest Endosc Clin N Am., 2016, 26(4):641-655.

Previous efforts using oral administration of naturally occurring omega-3 fatty acids to treat IBD have been unsuccessful. Lev-Tzion et al., Cochrane Database Syst. Rev., 2014, 28(2):CD006320; Cabre et al., Br. J. Nutri., 2012, Suppl 2:S240-252. This may be, at least in part, because these compounds are largely absorbed prior to reaching the lower small intestines, colon, and large intestines. In contrast, the inventors surprisingly found that a compound of Formula (I) not only reaches the distal small intestine and colon following oral administration, but accumulates in these regions of the intestine. Specifically, as provided in Biological Example 7, studies in rats found that after a single oral dose, Compound B accumulated in the caecum noted from 4 hours onwards to 1 day, and in the large intestine at 8 hours. As provided in Biological Example 8, Compound B is largely excreted via faeces, suggesting that Compound B accumulates in the intestine. This accumulation of a compound of Formula (I) in the small intestine and colon support use of these compounds for treating IBDs.

As provided in Biological Examples 9 and 10, mice with induced colitis showed a dose-dependent rescue from the colitis phenotypes of weight loss and decreased colon length when treated with a compound of Formula (I) compared to mice that were not administered a compound of Formula (I). In some embodiments, compounds of Formula (I) are for use in reducing weight loss in subjects with IBD compared with subjects with IBD not administered a compound of Formula (I). In some embodiments, compounds of Formula (I) are for use in maintaining body weight within 10% of initial body weight in subjects with IBD compared with subjects with IBD not administered a compound of Formula (I). In some embodiments, compounds of Formula (I) are for use in maintaining body weight within 5% of initial body weight in subjects with IBD compared with subjects with IBD not administered a compound of Formula (I). In some embodiments, compounds of Formula (I) are for use in reducing the decrease in colon length in subjects with IBD compared with subjects with IBD not administered a compound of Formula (I).

As shown in Biological Example 12, mice with induced colitis showed a dose-dependent rescue from colonic injury and inflammation when treated with a compound of Formula (I) based on histological scoring compared with mice that were not administered a compound of Formula (I). Further, and as shown in Biological Example 13, mice with induced colitis showed decreased colonic expression of key markers of inflammation when treated with Compound B. Specifically, Compound B reduced colonic expression of IL-6, IL-1b, S100A8, TNFα, and Reg3g, which are inflammatory cytokines and/or biomarkers associated with IBD.Eichele et al., World J. Gastroenterol., 2017, 23(33):6016-6029. In some embodiments, compounds of Formula (I) are for use in reducing intestinal inflammation in subjects with IBD compared with subjects with IBD not administered a compound of Formula (I). In some embodiments, compounds of Formula (I) are for use in reducing intestinal injury in patients with IBD compared with subjects with IBD not administered a compound of Formula (I).

In preferred embodiments, the disclosure provides a compound of Formula (I):

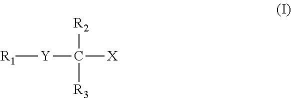

wherein R1 is selected from a C10-C22 alkenyl having 3-6 double bonds;
R2 and R3 are the same or different and are selected from a group of substituents consisting of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group, provided that R2 and R3 can be connected in order to form a cycloalkane like cyclopropane, cyclobutane, cyclopentane or cyclohexane, and provided that both R2 and R3 are not hydrogen;
X is a carboxylic acid or a derivative thereof, wherein the derivative is a carboxylate, such as a carboxylic ester; a glyceride; an anhydride; a carboxamide; a phospholipid; or a hydroxymethyl; or a prodrug thereof; and
Y is sulphur;
or a pharmaceutically acceptable salt, solvate, or solvate of such a salt;
for use in treating IBD, inducing remission of IBD, maintaining remission of IBD, reducing weight loss in patients with IBD, reducing the decrease in colon length in patients with IBD, reducing intestinal inflammation in patients with IBD, and/or reducing intestinal injury in patients with IBD.

In preferred embodiments, the disclosure provides a compound of Formula (I),
wherein R2 and R3 are independently chosen from a hydrogen atom or linear, branched, and/or cyclic C1-C6 alkyl groups, with the proviso that R2 and R3 cannot both be hydrogen atoms;
X is a carboxylic acid or a carboxylic ester; or a pharmaceutically acceptable salt, solvate, or solvate of such a salt; and
Y is sulphur;
for use in treating IBD, inducing remission of IBD, maintaining remission of IBD, reducing weight loss in patients with IBD, reducing the decrease in colon length in patients with IBD, reducing intestinal inflammation in patients with IBD, and/or reducing intestinal injury in patients with IBD.

In more preferred embodiments, the disclosure provides a compound of Formula (II):

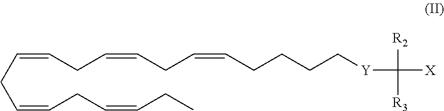

wherein R2 and R3 are the same or different and are selected from a group of substituents consisting of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group, provided that R2 and R3 can be connected in order to form a cycloalkane like cyclopropane, cyclobutane, cyclopentane or cyclohexane, and provided that both R2 and R3 are not hydrogen;
X is a carboxylic acid or a derivative thereof, wherein the derivative is a carboxylate, such as a carboxylic ester; a glyceride; an anhydride; a carboxamide; a phospholipid; or a hydroxymethyl; or a prodrug thereof; and
Y is sulphur;
for use in treating IBD, inducing remission of IBD, maintaining remission of IBD, reducing weight loss in patients with IBD, reducing the decrease in colon length in patients with IBD, reducing intestinal inflammation in patients with IBD, and/or reducing intestinal injury in patients with IBD.

In particularly preferred embodiments, the disclosure provides a compound of Formula (II),
wherein R2 and R3 are independently chosen from a hydrogen atom or linear, branched, and/or cyclic C1-C6 alkyl groups, with the proviso that both R2 and R3 cannot be hydrogen atoms;
X is a carboxylic acid or a carboxylic ester; or a pharmaceutically acceptable salt, solvate, or solvate of such a salt; and
Y is sulphur;
for use in treating IBD, inducing remission of IBD, maintaining remission of IBD, reducing weight loss in patients with IBD, reducing the decrease in colon length in patients with IBD, reducing intestinal inflammation in patients with IBD, and/or reducing intestinal injury in patients with IBD.

In some embodiments, the disclosure provides 2-ethyl-2-((5Z,8Z,11 Z,14Z,17Z)-icosa-5,8,11,14,17-pentaenylthio)butanoic acid:

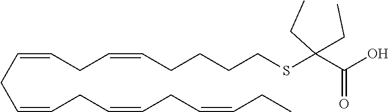 [SMILES: CC/C=C\C/C=C\C/C=C\C/C=C\C/C=C\CCCCSC(CC)(CC)C(=O)O]

for use in treating IBD, inducing remission of IBD, maintaining remission of IBD, reducing weight loss in patients with IBD, reducing the decrease in colon length in patients with IBD, reducing intestinal inflammation in patients with IBD, and/or reducing intestinal injury in patients with IBD.

The disclosed compounds are also suitable for use for the manufacture of a medicament for the described indications. For example, the disclosure provides for use of the compounds of Formula (I) for the manufacture of a medicament for treating IBD, such as ulcerative colitis, Crohn's disease, and indeterminate colitis. Likewise, the disclosure provides for use of the compounds of Formula (I) for the manufacture of a medicament for reducing intestinal inflammation in IBD, inducing remission of IBD, maintaining remission of IBD, reducing weight loss in subjects experiencing IBD symptoms, reducing decrease in colon length, reducing intestinal inflammation in subjects with IBD, and/or reducing intestinal injury in subjects with IBD.

In some embodiments, the disclosure provides use of at least two different active agents, a compound of Formula (I) or (II), and an additional active agent for treating IBD, inducing remission of IBD, maintaining remission of IBD, reducing weight loss in patients with IBD, reducing the decrease in colon length in patients with IBD, reducing intestinal inflammation in patients with IBD, and/or reducing intestinal injury in patients with IBD. Classes of drugs currently used to treat the symptoms of IBD include but are not limited to corticosteroids, aminosalicylates, immunosuppressants, small molecules and biologics. A non-limiting list of immunosuppressants include azathioprine (Azasan®, Imuran®), mercaptopurine (Purinethol®, Purixan®), cyclosporine (Gengraf®, Neoral®, Sandimmune®) and methotrexate (Trexall®). A non-limiting list of biologics include infliximab (Remicade®), adalimumab (Humira®), golimumab (Simponi®), natalizumab (Tysabri®), vedolizumab (Entyvio®) and ustekinumab (Stelara®). A non-limiting list of aminosalicylates include mesalamine (Asacol HD®, Delzicol®), balsalazide (Colazal®) and olsalazine (Dipentum®). A non-limiting list of corticosteroids include hydrocortisone, prednisolone, prednisone, and budesonide.

### EXAMPLES

### Synthesis Examples

### Example 1: Preparation of tert-butyl 2-((5Z,8Z,11Z,14Z,17Z)-icosa-5,8,11,14,17-pentaen-1-yloxy)butanoate

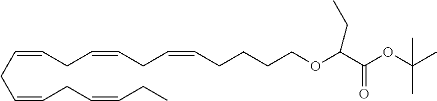 [SMILES: CC/C=C\C/C=C\C/C=C\C/C=C\C/C=C\CCCCOC(CC)C(=O)OC(C)(C)C]

Tetrabutylammonium chloride (0.55 g, 1.98 mmol) was added to a solution of (5Z,8Z,11Z,14Z,17Z)-icosa-5,8,11,14,17-pentaen-1-ol, (3.50 g, 12.1 mmol) in toluene (35 mL) at room temperature under nitrogen. An aqueous solution of sodium hydroxide (50% (w/w), 11.7 mL) was added under vigorous stirring at room temperature, followed by t-butyl 2-bromobutyrate (5.41 g, 24.3 mmol). The resulting mixture was heated to 50° C. and additional tbutyl 2-bromobutyrate was added after 1.5 hours (2.70 g, 12.1 mmol), 3.5 hours (2.70 g, 12.1 mmol) and 4.5 hours (2.70 g, 12.1 mmol) and stirred for 12 hours in total. After cooling to room temperature, ice water (25 mL) was added and the resulting two phases were separated. The organic phase was washed with a mixture of NaOH (5%) and brine, dried (MgSO₄), filtered and concentrated. The residue was purified by flash chromatography on silica gel using increasingly polar mixtures of heptane and ethyl acetate (100:0->95:5) as eluent. Concentration of the appropriate fractions afforded 1.87 g (36% yield) of the title compound as an oil. ¹H NMR (300 MHz, CDCl3): δ 0.85-1.10 (m, 6H), 1.35-1.54 (m, 11H), 1.53-1.87 (m, 4H), 1.96-2.26 (m, 4H), 2.70-3.02 (m, 8H), 3.31 (dt, 1H), 3.51-3.67 (m, 2H), 5.10-5.58 (m, 10H).

### Example 2: Preparation of 2-((5Z,8Z,11Z,14Z,17Z)-icosa-5,8,11,14,17-pentaenyloxy)butanoic acid (Compound A)

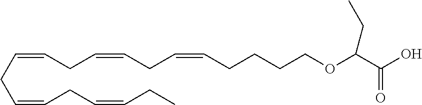 [SMILES: CC/C=C\C/C=C\C/C=C\C/C=C\C/C=C\CCCCOC(CC)C(=O)O]

tert-Butyl 2-((5Z,8Z,11Z,14Z,17Z)-icosa-5,8,11,14,17-pentaen-1-yloxy)butanoate (19.6 g, 45.5 mmol) was dissolved in dichloromethane (200 mL) and placed under nitrogen. Trifluoroacetic acid (50 mL) was added and the reaction mixture was stirred at room temperature for one hour. Water was added and the aqueous phase was extracted twice with dichloromethane. The combined organic extract was washed with brine, dried (Na₂SO₄), filtered and concentrated. The residue was subjected to flash chromatography on silica gel using increasingly polar mixtures of heptane, ethyl acetate and formic acid (90:10:1->80:20:1) as eluent. Concentration of the appropriate fractions afforded 12.1 g (71% yield) of the title compound as an oil. ¹H-NMR (300 MHz, CDCl₃): δ 0.90-1.00 (m, 6H), 1.50 (m, 2H), 1.70 (m, 2H), 1.80 (m, 2H), 2.10 (m, 4H), 2.80-2.90 (m, 8H), 3.50 (m, 1H), 3.60 (m, 1H), 3.75 (t, 1H), 5.30-5.50 (m, 10H); MS (electrospray): 373.2 [M−H]⁻.

### Example 3: Preparation of 2-ethyl-2-((5Z,8Z,1 1 Z,14Z,17Z)-icosa-5,8,1.1,14,17-pentaenylthio)butanoic Acid (Compound B)

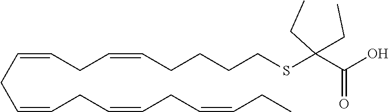 [SMILES: CC/C=C\C/C=C\C/C=C\C/C=C\C/C=C\CCCCSC(CC)(CC)C(=O)O]

NaOEt (21 weight percent in EtOH, 0.37 mL, 0.98 mmol) was added dropwise to a solution of 2-mercapto-2-ethyl butyric acid (0.08 g, 0.49 mmol) in dry EtOH (7 mL) held at 0° C. under inert atmosphere. The resulting mixture was stirred at 0° C. for 30 minutes before a solution of (5Z,8Z,1 1 Z,14Z,17Z)-icosa-5, 8,11,14,17-pentaenyl methanesulfonate (0.15 g, 0.41 mmol) in dry EtOH (3 ml) was added dropwise. The resulting turbid mixture was stirred at ambient temperature for 24 hours, poured into NH4Cl (sat.)(aq.) (15 ml), added 3M HCl to pH ˜2 before extracted twice with EtOAc (2×20 ml). The combined organic extracts were washed with brine (10 ml), dried (MgS04), filtrated and evaporated in vacuo. The residue was purified by flash chromatography on silica gel using a gradient of 10-25 percent ethyl acetate in heptane as eluent. Concentration of the appropriate fractions afforded 0.12 g (70 percent yield) of the title compound as oil. 1H-NMR (300 MHz, CDCl3): delta 0.88-1.02 (m, 9H), 1.45-1.58 (2×m, 4H), 1.72 (m, 2H), 1.82 (m, 2H) 2.09 (m, 4H), 2.53 (t, 2H), 2.76-2.86 (m, 8H), 5.29-5.39 (m, 10H. MS (electrospray): 417.3 [M−H]−.

### Example 4: Preparation of (4Z,7Z,10Z,13Z,16Z,19Z)-2,2-diethyldocosa-4,7,10,13,16,19-hexaenoic Acid

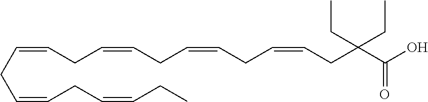 [SMILES: CC/C=C\C/C=C\C/C=C\C/C=C\C/C=C\C/C=C\CC(CC)(CC)C(=O)O]

Step a)

Butyllithium (38.6 ml, 0.62 mol, 1.6 M in hexane) was added dropwise to a stirring solution of diisopropylamine (9.1 ml, 0.65 mol) in dry THF (200 ml) under N₂ at 0° C. The resulting solution was stirred at 0° C. for 30 min. and cooled to −78° C. (Solution A). A solution of ethyl (4Z,7Z,10Z,13Z,16Z,19Z)-docosa-4,7,10,13,16,19-hexaenoate (DHA EE, 20.0 g, 0.56 mol) in dry THF (100 ml) was added dropwise to Solution A and the resulting mixture was stirred at −78° C. for 30 min. lodoethane (6.8 ml, 0.84 mol) was added and the reaction mixture was allowed to reach −10° C., then poured into water and extracted with hexane (2×). The combined organic phases were washed with 1 M HCl (aq), dried (Na2SO₄), filtered and evaporated in vacuo. The crude product was dissolved in in dry THF (100 ml) and added dropwise to a new batch of Solution A at −78° C. Iodoethane (6.8 ml, 0.84 mol) was added and the reaction mixture was allowed to reach ambient temperature. The mixture was stirred overnight, poured into water and extracted with hexane (2×). The combined organic phases were washed with 1 M HCl (aq), dried (Na₂SO₄), filtered and evaporated in vacuo. The crude product was purified by dry flash chromatography on silica gel eluting with heptane/EtOAc (99:1 followed by 98:2) to give 10.0 g (43% yield) of the titled compound as an oil; ¹H-NMR (200 MHz; CDCl₃) δ 0.83 (t, 6H), 0.94 (t, 3H), 1.28 (t, 3H), 1.63 (q, 4H), 2.10 (m, 2H), 2.34 (d, 2H), 2.8-3.0 (m, 10H), 4.15 (q, 2H), 5.3-5.6 (m, 12H); ¹³C-NMR (50 MHz; CDCl₃) δ 8.9, 14.7, 21.0, 23.1, 25.9, 26.0, 26.2, 27.4, 31.2, 50.1, 60.6, 125.5, 127.4, 128.3, 128.6, 128.9, 130.5, 132.4, 177.1; MS (electrospray); 413.3 [M+H], 435.3 [M+Na].

Step b)

Ethyl (4Z,7Z,10Z,13Z,16Z,19Z)-2,2-diethyldocosa-4,7,10,13,16,19-hexaenoate (2.42 g, 5.87 mmol) was dissolved in DMF (10 mL) and added thiophenol (0.63 mL, 6.17 mmol) and KOH (0.41 g, 6.17 mmol). The reaction mixture was stirred at 100° C. under N2 for 139 hours. The mixture was cooled, added 1M HCl (aq) and extracted with diethyl ether (4×). The organic layers were pooled, washed with brine, dried over MgSO4 and concentrated. The crude product was purified by flash chromatography (heptane:EtOAc 9:1, followed by 4:1 and then 7:3) to give 0.48 g (21% yield) of the title compound as an oil. 1H-NMR (200 MHz; CDCl3) δ 0.78 (t, 6H), 0.95 (t, 3H), 1.52-1.68 (m, 4H), 1.98-2.12 (m, 2H), 2.34 (d, 2H), 2.70-2.90 (m, 10H), 3.65 (s, 3H), 5.20-5.50 (m, 12H).

### Biological Examples

Evaluation of the Acute Effects of Compound a and Compound B Upon Active GLP-1 wconcentrations during an oral glucose tolerance test (OGTT) and at 24 h in lean male SPD Rats

To establish the acute effects of oral administration of Compound A and Compound B upon active GLP-1 and insulin concentrations, lean (about 300 g) male Sprague-Dawley (SPD) rats were divided into groups (n=6-8) and fed 74 and 84 mg/kg body weight of Compound A or Compound B respectively, either with or without concurrent administration of a dipeptidyl peptidase 4 (DPP-4) inhibitor 60 minutes prior to an oral glucose tolerance test (OGTT) as outlined below. Parallel groups receiving either corn oil alone (corn oil+vehicle, n=10) or corn oil and a DPP-4 inhibitor (“DPP-4 i”) (corn oil+(DPP-4 i), n=10) were included as controls. The DPP-4 inhibitor was linagliptin.

TABLE 1
In vivo study period

| Day 0 | −120 min | −60 min | 0 Min | 15 min | 30 min | 60 min | 120 min | 240 min | 480 min* | +24 hrs |
|---|---|---|---|---|---|---|---|---|---|---|
| OGTT | DPPPIV | Active | Active | Active | Active | Active | Active | Active | Active | Active |
| Semi- | Dosing/ | GLP-1 + | GLP-1 + | GLP-1 + | GLP-1 + | GLP-1 + | GLP-1 + | GLP-1 + | GLP-1 + | GLP-1 + |
| fasted | vehicle | Insulin + | Insulin + | Insulin | Insulin | Insulin | Insulin | Insulin + | Insulin + | Insulin |
| 60% | | Vehicle/ | Glucose | | | | | Vehicle/ | DPPIV | |
| | | Drug | 2 g/kg | | | | | Drug | Dosing/ | |
| | | dosing | | | | | | dosing + | vehicle | |
| | | | | | | | | Food | | |
| | | | | | | | | (ad lib) | | |

Samples were collected, as shown in Table 1, at 0, 15, 30 and 60 minutes for measurement of active GLP-1. A second oral dose of 74 and 84 mg/kg bodyweight of Compound A or Compound B respectively was administered at 240 minutes and ad lib feeding was initiated. A second dose of DPP-4 inhibitor was administered at 480 mins prior to lights out. Blood samples were collected at 24 h for measurement of active GLP-1 and insulin. All values are mean, figures depict mean values (SEM).

### Biological Example 1. Effects of Acute Feeding with Corn Oil+Vehicle, Corn Oil+DPP4 Inhibitor or Compound B+DPP4 Inhibitor, on Area Under the Curve (AUC) (0-60 Minutes) Glucose Stimulated Active GLP-1 (pg/ml)×Min in Lean SPD Rats

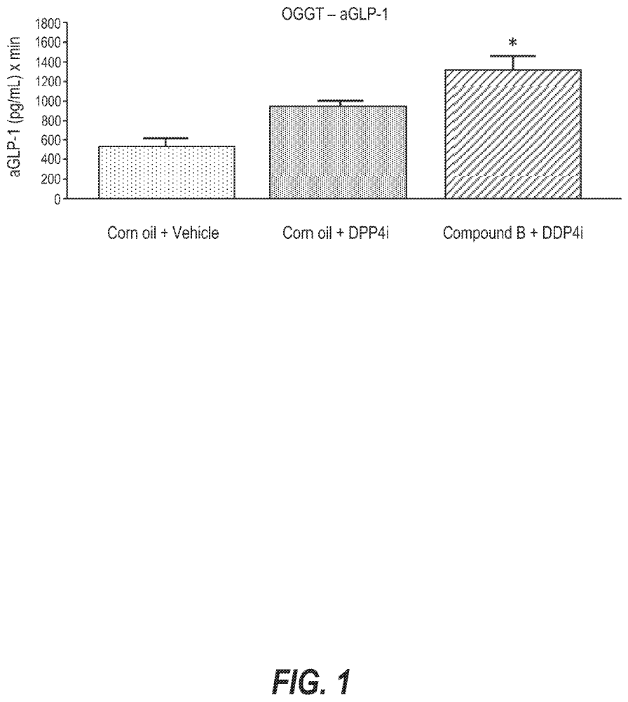

In combination with a DPP4 inhibitor, Compound B significantly (p<0.05) increased active GLP-1 (AUC 0-60 mins) concentrations versus corn oil+vehicle (>2-fold increase) whereas corn oil+DPP4 inhibitor alone had no significant effect versus corn oil alone. The results are presented in FIG. 1.

### Biological Example 2. Effects of Corn Oil+Vehicle, Corn Oil+DPP4 Inhibitor, Compound a Alone or Compound A+DPP4 Inhibitor on Active GLP-1 (pg/ml) at 24 h in Lean SPD Rats

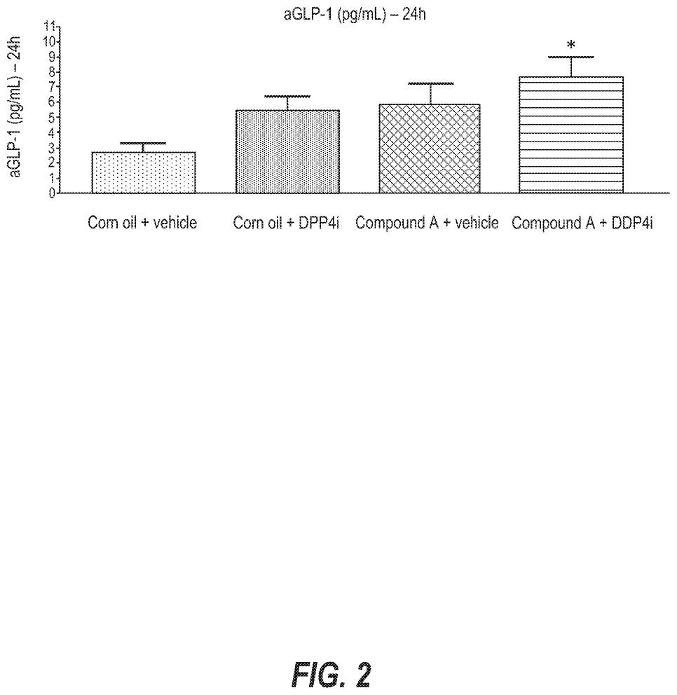

In combination with a DPP4 inhibitor, Compound A significantly (p<0.05) increased active 24 h GLP-1 concentrations versus corn oil+vehicle whereas corn oil+DPP4 inhibitor alone had no significant effect. The results are presented in FIG. 2.

### Biological Example 3. Effects of Corn Oil+Vehicle, Corn Oil+DPP4 Inhibitor, Compound B Alone or Compound B+DPP4 Inhibitor on Active GLP-1 (pg/ml) at 24 h in Lean SPD Rats

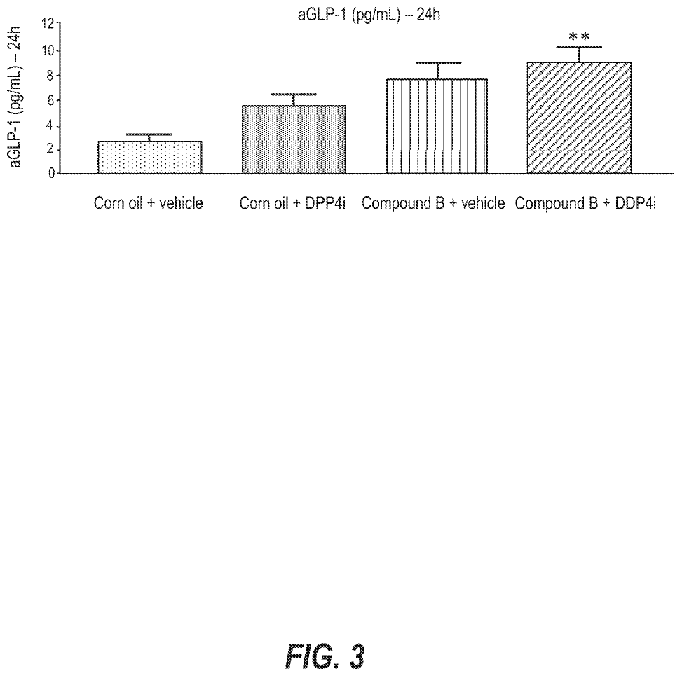

In combination with a DPP4 inhibitor, Compound B significantly (p<0.01) increased active GLP-1 concentrations versus corn oil+vehicle whereas corn oil+DPP4 inhibitor alone had no significant effect versus corn oil alone. The results are presented in FIG. 3.

### Biological Example 4. Effects of Corn Oil+Vehicle, Corn Oil+DPP4 Inhibitor, Compound a Alone or Compound A+DPP4 Inhibitor on Plasma Insulin (pg/ml) at 24 h in Lean SPD Rats

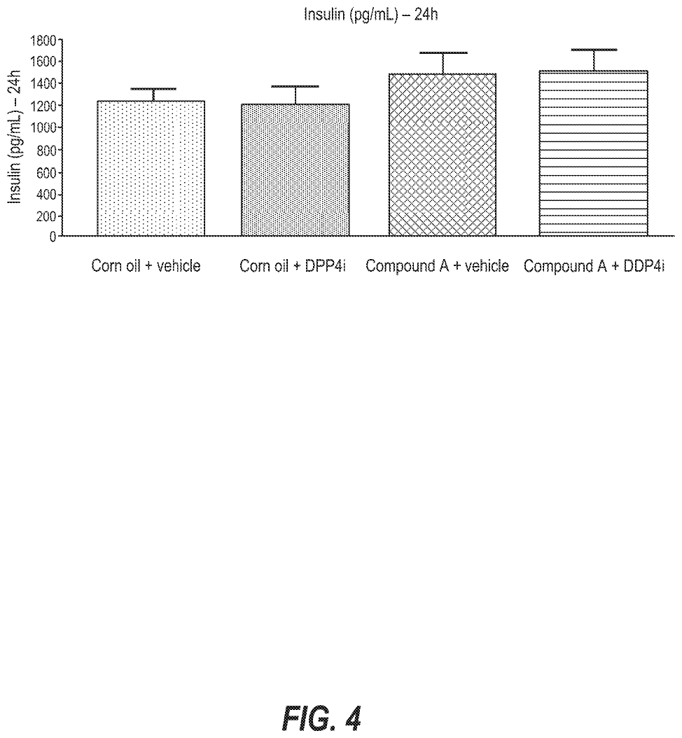

Either alone or in combination with a DPP4 inhibitor, Compound A increased insulin concentrations by 25% versus both corn oil+vehicle and corn oil+DPP4 inhibitor (non-significant). The results are presented in FIG. 4.

### Biological Example 5. Effects of Corn Oil+Vehicle, Corn Oil+DPP4 Inhibitor, Compound B Alone or Compound B+DPP4 Inhibitor on Plasma Insulin (pg/ml) at 24 h in SPD Lean Rats

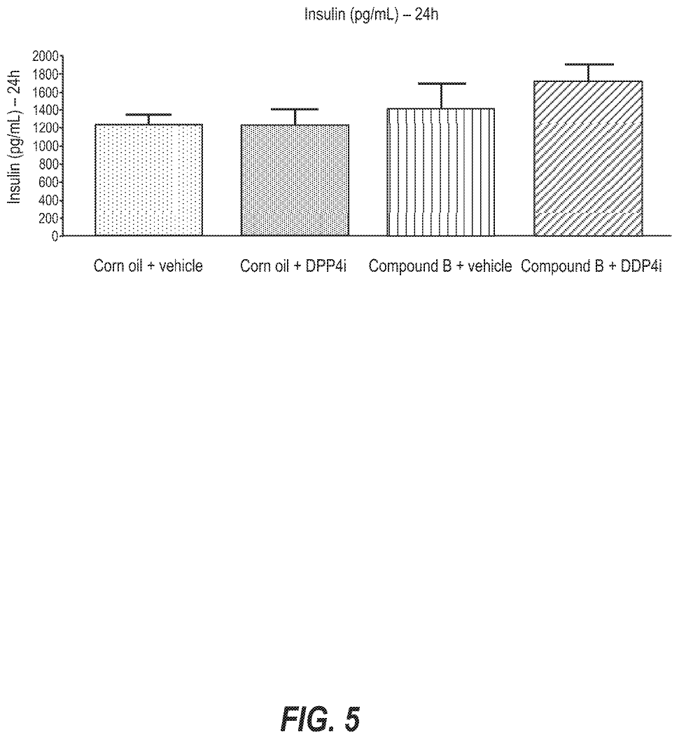

Either alone or in combination with a DPP4 inhibitor, Compound B increased insulin concentrations by 25% and 40% respectively versus both corn oil+vehicle and corn oil+DPP4 inhibitor (non-significant). The results are presented in FIG. 5.

### Biological Example 6. Effects of with Compound B or Compound a Versus Pioglitazone on Glucose Tolerance (0-120 Mins) in Ob/Ob Mice

This study was carried out to assess the effects of chronic treatment with Compound B or Compound A on glucose tolerance in a T2DM rodent model.

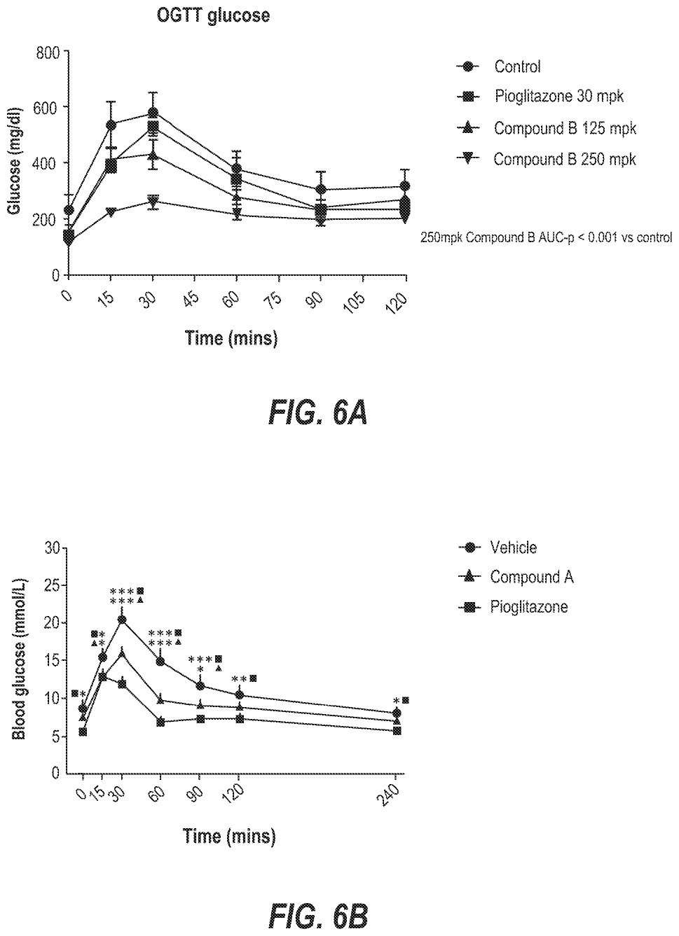

For assessing the effects of Compound B, B6.V-Lepob/Jrj mice (ob/ob) mice were administered Compound B at one of 2 doses, 125 and 250 mg/kg for 28 days. Eight-week old male ob/ob mice (8 per group) were treated once-daily via oral gavage with either Compound B (2 doses), pioglitazone (30 mg/kg) or vehicle and after 28 days were fasted for 5 hours before receiving a 2 g/kg oral glucose load. After the oral glucose load, plasma glucose was measured at multiple time points from and AUC (0-120 mins) for glucose was calculated. Both doses of Compound B improved glucose tolerance, with the 250 mg/kg dose inducing a potent and highly significant (p<0.001) reduction in AUC glucose (FIG. 6A).

For assessing the effects of Compound A, ob/ob mice were fed a high-fat diet (comprising 2% cholesterol, 40% fat (containing 18% trans-fatty acids), 20% fructose) for 15 weeks starting at age 5 weeks. The mice (10 per group) were administered Compound A (112 mg/kg), pioglitazone (30 mg/kg) or vehicle once-daily via diet. After 21 days the mice received a 2 g/kg oral glucose load. After the oral glucose load, plasma glucose was measured at multiple time points from 0-240 minutes. Compound A significantly improved glucose tolerance from 15 minutes through 90 minutes post-glucose load compared to vehicle (* p<0.05; ** p<0.01; *** p<0.001). Compound A also significantly reduced AUC glucose (p<0.01).

### Biological Example 7. Concentrations of Radioactivity in Intestinal Segments of Male Albino Rats after a Single Oral Administration of [14C]-Compound B at a Nominal Dose Level of 50 mg/kg Body Weight

This study was conducted to determine the intestinal tissue distribution of radioactivity in male albino rats following a single oral administration of [14C]-Compound B using quantitative whole-body autoradiography (QWBA). The tissue distribution in rats following a single oral dose of [14C]-Compound B at 50 mg/kg (Ca 5 MBq/kg) was studied by QWBA analysis up to 168 hours after dosing. Peak concentrations in the small intestinal mucosa occurred at 4 hours, with accumulation in the caecum noted from 4 hours onwards to 1 day, and at 8 hours in the large intestine, demonstrating the ability of Compound B to reach the distal small intestine and colon. The results are provided in Table 2.

TABLE 2

| | μg equivalents of Compound B/g of tissue | | | | | | |
|---|---|---|---|---|---|---|---|
| | Animal number and sex | | | | | | |
| | 6M | 5M | 4M | 3M | 7M | 8M | 9M |
| | Sampling time | | | | | | |
| Tissue | 1 hour | 2 hours | 4 hours | 8 hours | 1 day | 3 days | 7 days |
| Oesophageal wall | 5.83 | 1.72 | 1.29 | 5.86 | 2.25 | BLQ | BLQ |
| Stomach mucosa | 8.08 | 4.31 | 5.38 | 7.84 | 1.15 | BLQ | BLQ |
| (fundus) | | | | | | | |
| Stomach mucosa (non- | 17.3* | 28.1* | 300* | 252* | 3.87 | 0.253 | BLQ |
| fundic) | | | | | | | |
| Small intestine mucosa | 127* | 56.5* | 158* | 42.7 | 22.6 | 0.197 | BLQ |
| Caecum mucosa | 1.04 | 1.73 | 93.1* | 112* | 113* | 0.319 | 0.374 |
| Large intestine mucosa | 0.740 | 0.332 | 0.920 | 120* | 2.63 | 1.06 | 0.296 |
| Rectum mucosa | 0.417 | 0.883 | 1.03 | 3.57* | 0.870 | BLQ | 0.641 |
| Upper limit of | 1019 | 1019 | 1019 | 1019 | 1019 | 1019 | 1019 |
| quantification = | | | | | | | |
| Lower limit of | 0.175 | 0.175 | 0.175 | 0.175 | 0.175 | 0.175 | 0.175 |
| quantification = | | | | | | | |

*Measurement affected by high levels of radioactivity in adjacent contents
BLQ—Tissue concentration below lower limit of quantification

### Biological Example 8. Recovery of Radioactivity in the Excreta of Male Albino Rats after a Single Oral Administration of [14C]-Compound B at a Nominal Dose Level of 50 mg/kg Body Weight

To assess Compound B clearance via urine versus feces, the excretion pattern of a single oral dose of [14C]-Compound B was determined in male albino rats. The excretion pattern was similar in each animal and quantitative recoveries of radioactivity were obtained (101%). The total excretion of radioactivity following oral administration was >95% within the first 48 hours. Excretion via the urine accounted for 12% of the administered dose. Faecal elimination was 86% following oral dosing, suggesting that a large amount of [14C]-Compound B-related material was excreted without being absorbed. Table 3 provides the results from the Excretion Balance Investigation.

TABLE 3

| | % Recovery of Administered Dose |
|---|---|
| Sample | (Mean) |
| Urine (0-168 h) | 12.4 |
| Faeces (0-168 h) | 85.8 |
| Cage wash* (0-168 h) | 1.27 |
| Cage debris (0-168 h) | 0.038 |
| Carcass (168 h) | 1.14 |
| Mean total radioactivity (0-168 h) | 101 |

Mean-n = 3
*includes final cage wash

Evaluation of Effects of Compound B on Intestinal Inflammation in DSS-Induced Colitis Mice

The dextran sodium sulphate-induced (DSS)-induced colitis model is well known in the art as a reproducible chemical induction of intestinal inflammation animal model. See, e.g., Eichele et al., World J Gastroenterol, 2017, 23(33):6016-6029; Randhawa et al., Korean J. Physiol. Pharmacol. (2014) 18:279-288; Jurjus et al., J. Pharmacol. Toxicol., Methods, 2004, 50:81-92; Gaudio et al., Dig. Dis. Sci., 1999, 44:1458-1475. The DSS-induced colitis model morphologically and symptomatically resembles epithelial damage seen in human IBD, and thus has become the most extensively employed experimental model of intestinal inflammation. Okayasu et al., Gastroenterology, 1990, 98:694-702; Kawada et al., World J. Gastroenterol. 2007, 13:5581-5593. The DSS-induced colitis model is most similar to ulcerative colitis in humans, but also has many similarities to Crohn's disease.

DSS is a water soluble, negatively charged sulfated polysaccharide with a highly variable molecular weight ranging from 5 to 1400 kDa. Murine colitis results from administration of about 1% to 3% DSS to the drinking water of a mouse strain susceptible to DSS-induced colitis. Without being bound by theory, the sulfated polysaccharide may not directly induce intestinal inflammation, but may instead act as a direct chemical toxin to colonic epithelium resulting in epithelial cell injury. It is thought that DSS disrupts the intestinal epithelial monolayer lining, leading to the entry of luminal bacteria and associated antigens into the mucosa and allowing the dissemination of proinflammatory intestinal contents into underlying tissue. DSS at a size range of about 40-50 kDa added to sterilized drinking water has been shown to penetrate the mucosal membrane of the intestine. Perse et al., J. Biomed. Biotechnol., 2012:718617.

The C56BL/6J mouse is a strain susceptible to DSS-induced colitis. To evaluate the efficacy and dosage of Compound B in treatment of DSS-induced colitis, inflammation was induced in 30 9-week old C56BL/6J mice by adding 1.5% DSS to the drinking water for 7 days. The mice were fed a standard chow diet that consisted of 30 weight percent wheat. The mice were divided into three groups of 10, and for each day of DSS administration each group was administered via oral gavage either (1) 100 μL corn oil per day (Control), (2) 126 mg/kg Compound B (dissolved in 100 μL corn oil) per day (“Compound B—Low” or “Compound B—L”), or (3) 252 mg/kg Compound B (dissolved in 100 μL corn oil) per day (“Compound B—High” or “Compound B—H”). Following the 7-day period of DSS induction, mice were sacrificed and their intestinal tissue was used for histopathological and gene expression analysis.

### Biological Example 9

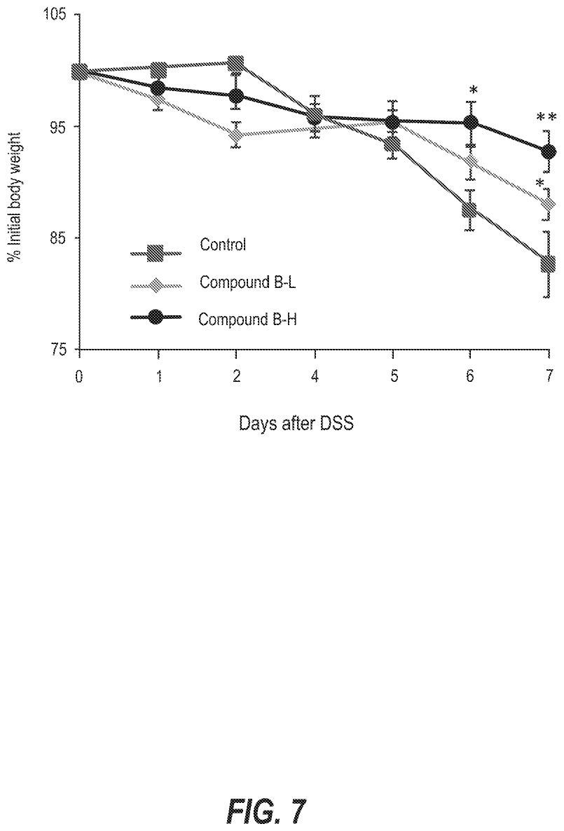

To assess the efficacy of Compound B in the treatment of DSS-induced colitis in mice, the body weight of mice was monitored. Weight loss is an indicator of colitis severity. As shown in FIG. 7, mice fed with 1.5% DSS in the drinking water showed a progressive loss of body weight. Compared to the control group, mice treated with Compound B showed a dose-dependent reduction in weight loss. The difference in weight loss between the control vs. treated groups was statistically significant after 6 days of DSS induction for the Compound B—High group and was statistically significant after 7 days for both the Compound B—High and Compound B—Low groups.

### Biological Example 10

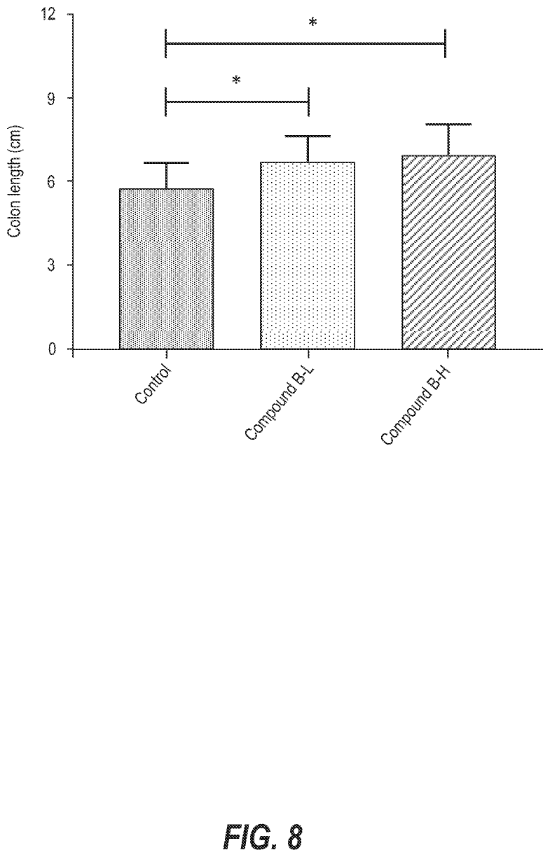

To assess the efficacy of Compound B in the treatment of DSS-induced colitis in mice, colon length of test mice was measured. Colon length correlates inversely with inflammation. As shown in FIG. 8, mice treated with Compound B at both the low and high doses showed a significant increase in colon length compared to control.

### Biological Example 11

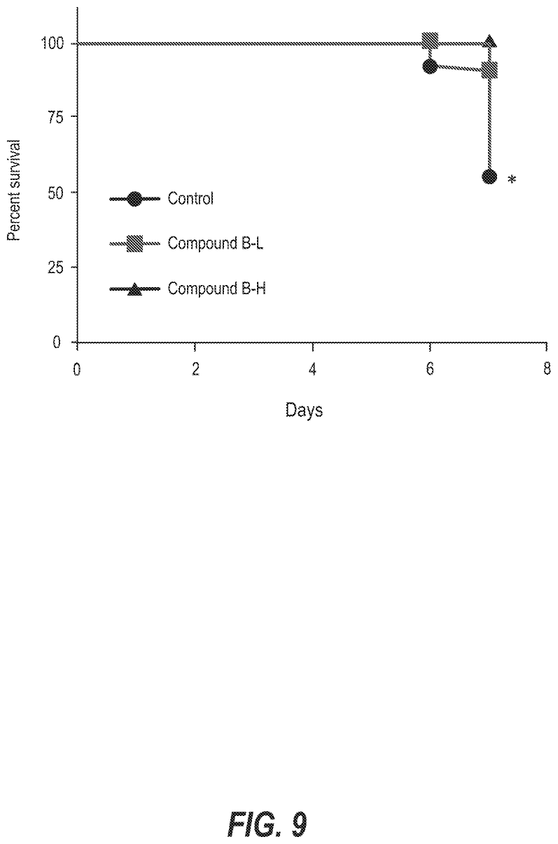

As shown in FIG. 9, mice treated with Compound B showed a dose-dependent increase in survival rate compared to control. 50% (n=5) of untreated mice in the control group were alive 7 days after colitis was induced, compared to 90% (n=9) of mice treated with the low dose of Compound B, and 100% (n=10) of mice treated with the high dose of compound B. Deaths in the control group were due to sepsis and severe colon inflammation. Thus, Compound B has a statistically significant effect on survival rate in colitis mice.

### Biological Example 12

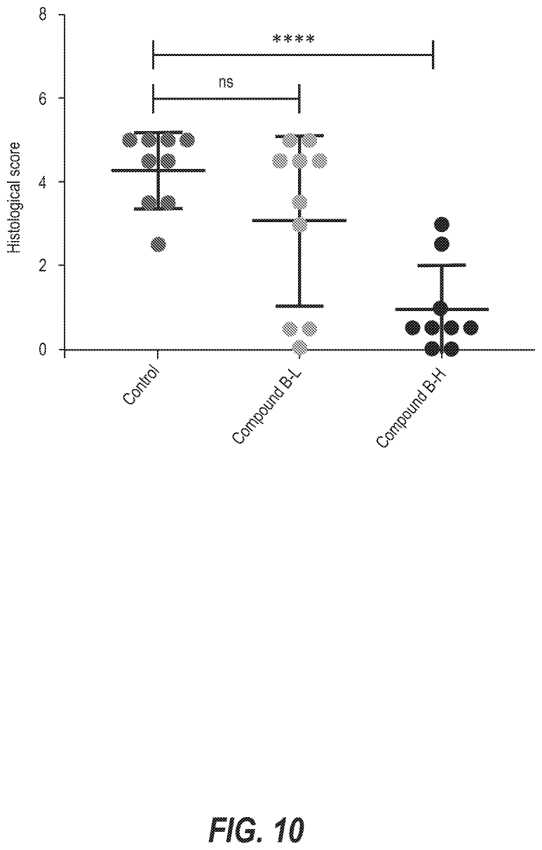

Histopathological analysis was performed on sections of formalin-fixed paraffin embedded tissue after hematoxylin and eosin (H&E) staining. Colonic samples were analyzed by histopathology for assignment of scores for colitis activity as described in Neurath et al., J. Exp. Med., 2002, 195:1129-1143. Briefly, the degrees of inflammation and epithelial and mucosal injury on microscopic cross-sections of the colon were graded semi-quantitatively from 0 to 4. For inflammation, a score of 0=no evidence for inflammation; 1=low level of inflammation with scattered infiltrating mononuclear cells (1-2 foci only); 2=moderate inflammation with multiple foci; 3=high level of inflammation with increased vascular density and marked wall thickening; and 4=maximal severity of inflammation with transmural leukocyte infiltration and loss of goblet cells. For injury, a score of 0=no epithelial injury; 1=occasional epithelial lesion; 2=1-2 foci of ulcerations; and 3=extensive ulcerations. Small bowel sections were taken from uninduced (i.e., no DSS) animals as an additional control and showed no evidence of inflammation. As shown in FIG. 10, histological scores of samples from mice treated with the high dose of Compound B were significantly lower than that from untreated mice. Both inflammation and epithelial and mucosal injury were lower in mice treated with the high dose of Compound B than in unentreated mice.

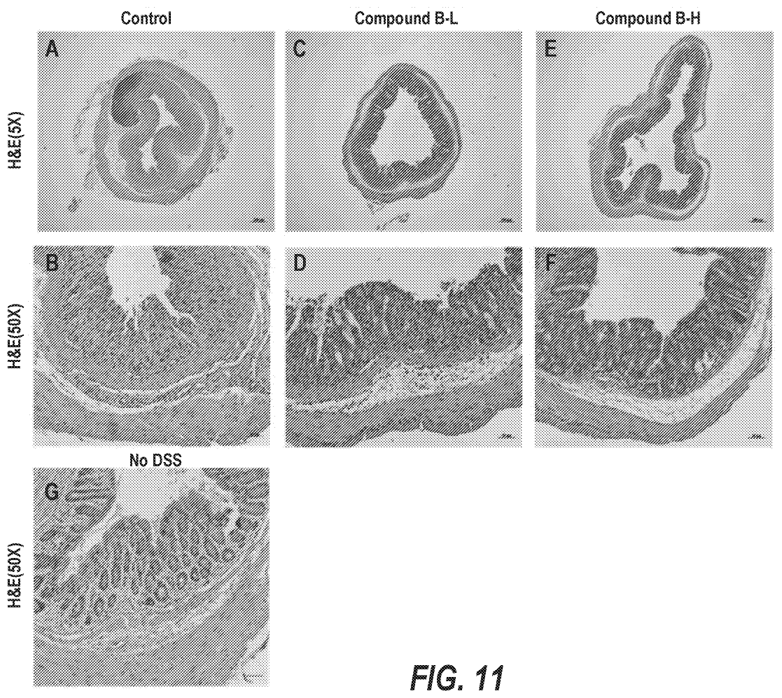

Representative histological cross-sections of the colons of DSS-induced mice, as well as from an uninduced (i.e., no DSS) mouse, are shown in FIG. 11. In DSS-induced control mice (FIGS. 11A and B), histological cross-sections show effacement of the villus-crypt architecture, edema and inflammatory infiltration/foci of the lamina propria and muscularis mucosae, intestinal epithelial cell shedding, and loss of the protective mucus layer (orange colour). In treated mice (FIGS. 11C-F), histology shows a dose-dependent attenuation of inflammatory infiltrates and edema, and reconstitution of villus architecture and mucus later to near normal morphology. In comparison, histological cross-sections of colon treated with a high dose of Compound B shows almost complete rescue, with morphology resembling that of colon from a mouse that has not been administered DSS (FIG. 11G). The scale bars for FIGS. 11A, C and E are 200 μm. The scale bars for FIGS. 11 B, D, F, and G are 50 μm.

### Biological Example 13

To evaluate the effect of treatment with Compound B on the levels of proinflammatory cytokines and biomarkers, total RNA was extracted from small and large intestinal tissue samples (>100 mg). cDNA was synthesized by reverse transcription and analyzed by real-time PCR. Results were normalized to the level of the housekeeping gene hypoxanthine guanine phosphoribosyltransferase (HPRT). The relative mRNA expression of the tested gene relative to HPRT expression was calculated using the 2^{-ΔΔCt} method as described in Pickert et al., J. Exp. Med., 2009, 206:1465-1472. Interleukin 6 (IL6), IL1b, calgranulin-A (S100A8), and tumor necrosis factor α (TNFα) have been implicated as mediators of IBD, including both ulcerative colitis and Crohn's disease. IL6, IL1b, and calgranulin-A are prominently expressed in inflammatory macrophages. IL22-dependent regenerating islet-derived 3 gamma (Reg3g) is induced in response to inflammation in epithelial cells. IL17 is secreted by Th17 T helper cells and innate lymphoid cells type 3 (ILC3).

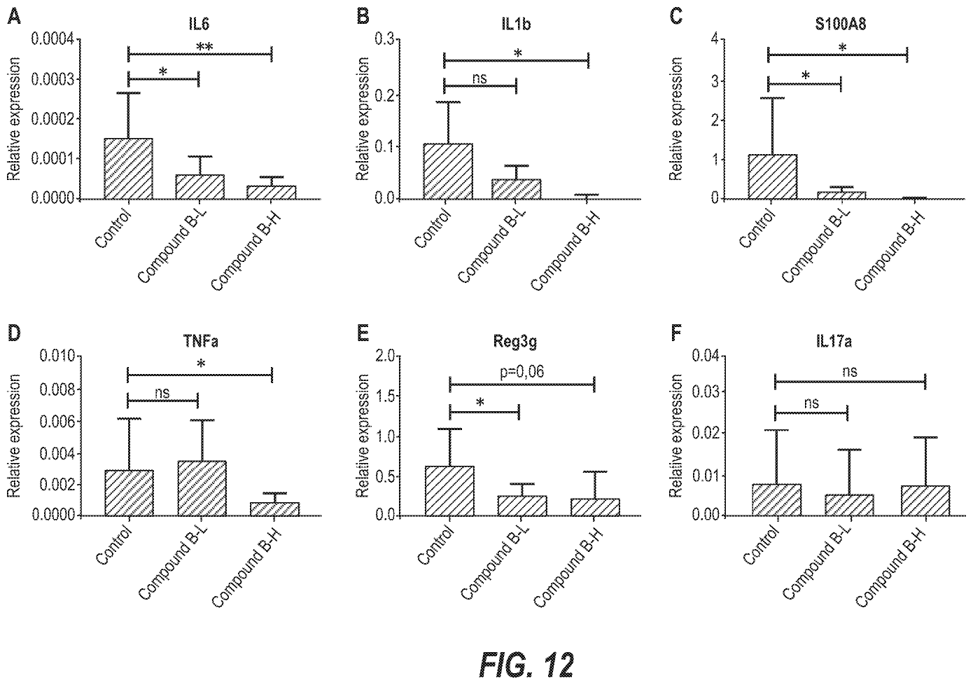

As shown in FIG. 12, mRNA levels of IL6, IL1b, S100A8, TNFα and Reg3g showed a dose-dependent decrease in Compound B treated mice compared to untreated mice, consistent with rescue from colitis and reduction in inflammation. The lack of change in expression of IL17a in response to Compound B treatment is consistent with protection against IBD. Taken altogether, the results show that Compound B may have a clinically beneficial effect on colitis and other inflammatory bowel disorders, such as Crohn's disease and indeterminate colitis.

### Biological Example 14

To evaluate the effects of chronic treatment with Compound A in a rodent model of T2DM, 6-8 week old male ob/ob mice were administered one of three doses of Compound A (15 mg/kg bw/d; 45 mg/kg bw/d; 135 mg/kg bw/d) via diet admix, pioglitazone (30 mg/kg bw/d) via diet admix, fenofibrate (100 mg/kg bw/d) via diet admix, or were untreated (control) for 5 weeks (10 mice per group). Mice were fed a standard low-fat (7% w/w fat) diet. After 4-weeks, the mice were fasted for 4 hours and the effects of Compound A were assessed. Assessment of the effects of Compound A included basal levels of blood glucose, plasma insulin, HbA1c levels, and homeostatic model assessment of insulin resistance (HOMA-IR). HOMA-IR is an assessment of insulin resistance and is calculated by the following formula: fasting insulin (micro U/L)×fasting glucose (nmol/L)/22.5. The effects of the 135 mg/kg dose of Compound A are provided in Table 4.

TABLE 4

| | Control | Compound A | Fenofibrate | Pioglitazone |
|---|---|---|---|---|
| Body weight (g) | 54.5 ± 1.5 | 55.0 ± 1.1 | 56.5 ± 1.4 | 62.3 ± 1.6* |
| Food intake | 6.8 ± 0.5 | 6.1 ± 0.3 | 4.4 ± 0.2 | 6.2 ± 0.1 |
| (g/m/d) | | | | |
| Blood glucose | 380.8 ± 26.3 | 191.5 ± 6.1* | 220.3 ± 8.1* | 147.5 ± 3.1* |
| (mg/dL) | | | | |
| Plasma insulin | 73.2 ± 13.6 | 17.6 ± 6.8* | 35.5 ± 9.0* | 5.7 ± 0.8* |
| (ng/mL) | | | | |
| Blood HbA1c | 8.4 ± 0.4 | 4.5 ± 0.1* | 4.8 ± 0.2* | 3.8 ± 0.1* |
| (%) | | | | |
| HOMA-IR | 65.7 ± 9.8 | 8.6 ± 3.6* | 20.3 ± 5.9* | 2.1 ± 0.3* |
| Plasma | 4.3 ± 0.4 | 3.8 ± 0.4 | 5.0 ± 0.4 | 19.3 ± 1.8* |
| adiponectin | | | | |
| (μg/mL) | | | | |
| Plasma ALT | 271.4 ± 18.8 | 180.6 ± 14.6* | 224.0 ± 27.4 | 257.3 ± 15.5 |
| (U/L) | | | | |

Data represent mean ± standard error of the mean.
*p < 0.05 versus control.

After 5 weeks, an oral glucose (2 g/kg) tolerance test was performed after a 4 hour fast. Compound A showed a dose-dependent response in lowering glucose levels.

TABLE 5

| | Plasma glucose (mM) | | | | | | |
|---|---|---|---|---|---|---|---|
| | | | | | | | AUC |
| | 0 min | 15 min | 30 min | 45 min | 60 min | 120 min | (0-120 min) |
| Control | 18.0 ± 6.4 | 43.8 ± 5.4 | 42.8 ± 6.2 | 38.3 ± 8.0 | 36.6 ± 10.6 | 26.7 ± 10.3 | 2026 ± 348 |
| 15 mg/kg bw/d | 11.4 ± 1.5*^{†} | 37.5 ± 5.7* | 34.4 ± 9.3* | 28.0 ± 9.0* | 25.5 ± 10.1* | 19.6 ± 7.8 | 1762 ± 807 |
| Compound A | | | | | | | |
| 45 mg/kg bw/d | 13.3 ± 3.7* | 36.5 ± 8.0 | 30.1 ± 6.7* | 24.5 ± 7.4*^{†} | 21.0 ± 6.5*^{†} | 15.3 ± 3.5* | 1112 ± 588* |
| Compound A | | | | | | | |
| 135 mg/kg bw/d | 9.2 ± 2.0*^{†} | 27.7 ± 5.6*^{†} | 22.1 ± 6.5*^{†} | 14.6 ± 3.1*^{†} | 13.4 ± 2.3*^{†} | 12.8 ± 1.8*^{†} | 816 ± 281* |
| Compound A | | | | | | | |
| Fenofibrate | 15.4 ± 4.7 | 35.9 ± 5.3* | 35.9 ± 6.4* | 30.6 ± 5.2* | 28.2 ± 6.0* | 18.7 ± 5.3* | 1416 ± 400* |
| Pioglitazone | 7.8 ± 0.8* | 24.2 ± 6.0* | 18.5 ± 14.4* | 17.3 ± 14.0* | 15.2 ± 10.7* | 10.9 ± 3.5* | 879 ± 1023* |

Data represent mean ± standard deviation.
*p < 0.05 versus control;
^{†}p < 0.05 for Compound A versus fenofibrate.

1.-77. (canceled)

78. A method for reducing basal and/or postprandial hyperglycemia and/or increasing postprandial plasma insulin concentration in a subject in need thereof, the method comprising administering to the subject a pharmaceutically effective amount of a compound of Formula (I): 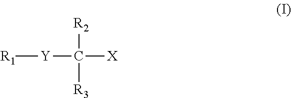
wherein R1 is selected from a C10-C22 alkenyl having 3-6 double bonds;
R2 and R3 are the same or different and are selected from a group of substituents consisting of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group, provided that R2 and R3 can be connected in order to form a cycloalkane like cyclopropane, cyclobutane, cyclopentane or cyclohexane, and provided that both R2 and R3 are not hydrogen;
X is a carboxylic acid or a derivative thereof, wherein the derivative is a carboxylate, such as a carboxylic ester; a glyceride; an anhydride; a carboxamide; a phospholipid; or a hydroxymethyl; or a prodrug thereof;
Y is oxygen, sulphur, sulfoxide or sulfone;
or a pharmaceutically acceptable salt, solvate, or solvate of such a salt

79. The method according to claim 78, wherein R1 is a C18-C22 alkenyl having 3-6 double bonds, and wherein one double bond is in the omega-3 position.

80. The method according to claim 78, wherein R2 and R3 are independently selected from the group of a hydrogen atom and linear, branched, and/or cyclic C1-C6 alkyl groups.

81. The method according to claim 78, wherein Y is oxygen or sulphur.

82. The method according to claim 78, wherein the compound is a compound of Formula (II):

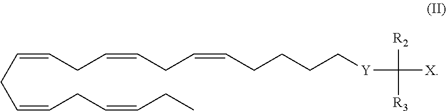

83. The method according to claim 78, wherein basal and/or postprandial hyperglycemia is reduced.

84. The method according to claim 83, wherein the subject has type 2 diabetes.

85. The method according to claim 83, wherein postprandial plasma glucose levels are decreased at 15 minutes and/or 30 minutes postprandial.

86. The method according to claim 78, wherein postprandial plasma insulin concentration is increased.

87. The method according to claim 78, wherein the method further comprises administering a pharmaceutically effective amount of a DPP-4 inhibitor.

88. A method for treating IBD in a subject in need thereof, the method comprising administering to the subject a pharmaceutically effective amount of a compound of Formula (I): 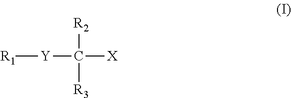
wherein R1 is selected from a C10-C22 alkenyl having 3-6 double bonds;
R2 and R3 are the same or different and are selected from a group of substituents consisting of a hydrogen atom, a hydroxy group, an alkyl group, a halogen atom, an alkoxy group, an acyloxy group, an acyl group, an alkenyl group, an alkynyl group, an aryl group, an alkylthio group, an alkoxycarbonyl group, a carboxy group, an alkylsulfinyl group, an alkylsulfonyl group, an amino group, and an alkylamino group, provided that R2 and R3 can be connected in order to form a cycloalkane like cyclopropane, cyclobutane, cyclopentane or cyclohexane, and provided that both R2 and R3 are not hydrogen;
X is a carboxylic acid or a derivative thereof, wherein the derivative is a carboxylate, such as a carboxylic ester; a glyceride; an anhydride; a carboxamide; a phospholipid; or a hydroxymethyl; or a prodrug thereof;
Y is oxygen, sulphur, sulfoxide or sulfone;
or a pharmaceutically acceptable salt, solvate, or solvate of such a salt

89. The method according to claim 88, wherein Y is sulphur.

90. The method according to claim 88, wherein R1 is a C18-C22 alkenyl having 5 or 6 methylene interrupted double bonds, wherein the first double bond is between the third and fourth carbons from the omega end.

91. The method according to claim 88, wherein R2 and R3 are independently chosen from a hydrogen atom and linear, branched, and/or cyclic C1-C6 alkyl groups.

92. The method according to claim 88, wherein X is a carboxylic acid or a carboxylic ester.

93. The method according to claim 88, wherein the compound is a compound of Formula (II):

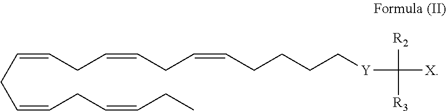

94. The method according to claim 88, wherein R2 and R3 are ethyl groups.

95. The method according to claim 88, wherein the compound is 2-ethyl-2-((5Z,8Z,11 Z,14Z,17Z)-icosa-5,8,11,14,17-pentaenylthio)butanoic acid:

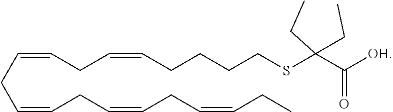 [SMILES: CC/C=C\C/C=C\C/C=C\C/C=C\C/C=C\CCCCSC(CC)(CC)C(=O)O]

96. The method according to claim 88, wherein the IBD is chosen from ulcerative colitis, Crohn's disease, and indeterminate colitis.

97. The method according to claim 88, wherein the method comprises reducing intestinal inflammation.

98. The method according to claim 88, wherein the method comprises reducing intestinal injury.

99. The method according to claim 88, wherein the method further comprises administering a co-administering an additional active agent.
